SEQUENCE LISTING (109 sequences)

SEQ ID NO: 1 - protein, 107 aa, homo sapiens
LLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 2 - protein, 106 aa, Homo sapiens [sequence omitted]

SEQ ID NO: 3 - DNA, 321 bp, Homo sapiens
ctcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 4 - DNA, 324 bp, Homo sapiens
gggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 5 - protein, 214 aa, Homo sapiens
LLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 645 bp, Homo sapiens
ctcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 7 - DNA, 57 bp, Homo sapiens
atggaattggggctgagctgggttttccttgttgctattttagaaggtgtccagtgt

SEQ ID NO: 8 - DNA, 87 bp, Mus musculus
ctgtgggccaccgccagcaccttcatcgtgctgttcctgctgagcctgttctacagcaccaccgtgaccctgttcaaagtgaaatag

SEQ ID NO: 9 - DNA, 87 bp, homo sapiens; identical to SEQ ID NO: 8
  misc_feature (1)..(87): Coding sequence for the membrane IgM Transmembrane region

SEQ ID NO: 10 - DNA, 150 bp, homo sapiens
gctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttag

SEQ ID NO: 11 - DNA, 102 bp, homo sapiens
ggcaccaccgacgccgcgcacccggggcggtccgtggtccccgcgttgcttcctctgctggccgggaccctgctgctgctggagacggccactgctccctga

SEQ ID NO: 12 - DNA, 979 bp, Artificial sequence
  synthetic
ggcttggggatatccaccatggagacagacacactcctgctatgggtactgctgctctgggttccaggttccactggtgactatccatatgatgttccagattatgctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaagaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggcggccgctcgag

SEQ ID NO: 13 - DNA, 63 bp, Mus musculus
atggagacagacacactcctgctatgggtactgctgctctgggttccaggttccactggtgac

SEQ ID NO: 14 - DNA, 27 bp, homo sapiens
tatccatatgatgttccagattatgct

SEQ ID NO: 15 - DNA, 675 bp, homo sapiens
actcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 16 - DNA, 33 bp, homo sapiens
gaacaaaaactcatctcagaagaggatctgaat

SEQ ID NO: 17 - DNA, 150 bp, homo sapiens; identical to SEQ ID NO: 10

SEQ ID NO: 18 - DNA, 916 bp, Artificial sequence
  synthetic
ggcttggggatatccaccatggaattggggctgagctgggttttccttgttgctattttagaaggtgtccagtgttatccatatgatgttccagattatgctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaagaacaaaaactcatctcagaagaggatctgaatctgtgggccaccgccagcaccttcatcgtgctgttcctgctgagcctgttctacagcaccaccgtgaccctgttcaaagtgaaataggcggccgctcgagatacga

SEQ ID NO: 19 - DNA, 931 bp, Artificial sequence
  synthetic
ggcttggggatatccaccatggaattggggctgagctgggttttccttgttgctattttagaaggtgtccagtgttatccatatgatgttccagattatgctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaagaacaaaaactcatctcagaagaggatctgaatggcaccaccgacgccgcgcacccggggcggtccgtggtccccgcgttgcttcctctgctggccgggaccctgctgctgctggagacggccactgctccctgagcggccgctcgagatacga

SEQ ID NO: 20 - protein, 108 aa, homo sapiens
LLGGPSVFLFPPKPKDTLMISRTPEVTCVVVDVSHEDPEKVKFNWYVDGVEVHNAKTKPREEQYNSTYRVVSVLTVLHQDWLDGKEYKCKVSNKALPAPIEKTISKAK

SEQ ID NO: 21 - protein, 107 aa, homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 4 aa, homo sapiens
LLGG

SEQ ID NO: 23 - protein, 6 aa, homo sapiens
DVSHED

SEQ ID NO: 24 - protein, 3 aa, homo sapiens
NST

SEQ ID NO: 25 - protein, 7 aa, homo sapiens
KALPAPI

SEQ ID NO: 26 - DNA, 63 bp, homo sapiens
ccaccgtgcccagcacctgaagggctggggggaccgtcagtcttcctcttccccccaaaaccc

SEQ ID NO: 27 - DNA, 63 bp, homo sapiens
ccaccgtgcccagcacctgaactcggggggggaccgtcagtcttcctcttccccccaaaaccc

SEQ ID NO: 28 - DNA, 63 bp, homo sapiens
ccaccgtgcccagcacctgaaaacctggggggaccgtcagtcttcctcttccccccaaaaccc

SEQ ID NO: 29 - DNA, 63 bp, homo sapiens
ccaccgtgcccagcacctgaactcaacgggggaccgtcagtcttcctcttccccccaaaaccc

SEQ ID NO: 30 - DNA, 63 bp, homo sapiens
ccaccgtgcccagcacctgaactcctgaacggaccgtcagtcttcctcttccccccaaaaccc

SEQ ID NO: 31 - DNA, 63 bp, homo sapiens
ccaccgtgcccagcacctgaactcctggggaacccgtcagtcttcctcttccccccaaaaccc

SEQ ID NO: 32 - DNA, 63 bp, homo sapiens
ccaccgtgcccagcacctgaagacctggggggaccgtcagtcttcctcttccccccaaaaccc

SEQ ID NO: 33 - DNA, 63 bp, homo sapiens
ccaccgtgcccagcacctgaactcgacgggggaccgtcagtcttcctcttccccccaaaaccc

SEQ ID NO: 34 - DNA, 63 bp, homo sapiens
ccaccgtgcccagcacctgaactcctggacggaccgtcagtcttcctcttccccccaaaaccc

SEQ ID NO: 35 - DNA, 63 bp, homo sapiens
ccaccgtgcccagcacctgaactcctgggggacccgtcagtcttcctcttccccccaaaaccc

SEQ ID NO: 36 - DNA, 63 bp, homo sapiens
ccaccgtgcccagcacctgaacacctggggggaccgtcagtcttcctcttccccccaaaaccc

SEQ ID NO: 37 - DNA, 63 bp, homo sapiens
ccaccgtgcccagcacctgaactccacgggggaccgtcagtcttcctcttccccccaaaaccc

SEQ ID NO: 38 - DNA, 63 bp, homo sapiens
ccaccgtgcccagcacctgaactcctgcacggaccgtcagtcttcctcttccccccaaaaccc

SEQ ID NO: 39 - DNA, 63 bp, homo sapiens
ccaccgtgcccagcacctgaactcctggggcacccgtcagtcttcctcttccccccaaaaccc

SEQ ID NO: 40 - DNA, 63 bp, homo sapiens
ccaccgtgcccagcacctgaatggctggggggaccgtcagtcttcctcttccccccaaaaccc

SEQ ID NO: 41 - DNA, 63 bp, homo sapiens
ccaccgtgcccagcacctgaactctgggggggaccgtcagtcttcctcttccccccaaaaccc

SEQ ID NO: 42 - DNA, 63 bp, homo sapiens
ccaccgtgcccagcacctgaactcctgtggggaccgtcagtcttcctcttccccccaaaaccc

SEQ ID NO: 43 - DNA, 63 bp, homo sapiens
ccaccgtgcccagcacctgaactcctggggtggccgtcagtcttcctcttccccccaaaaccc

SEQ ID NO: 44 - DNA, 63 bp, homo sapiens
ccaccgtgcccagcacctgaaatcctggggggaccgtcagtcttcctcttccccccaaaaccc

SEQ ID NO: 45 - DNA, 63 bp, homo sapiens
ccaccgtgcccagcacctgaactcatcgggggaccgtcagtcttcctcttccccccaaaaccc

SEQ ID NO: 46 - DNA, 63 bp, homo sapiens
ccaccgtgcccagcacctgaactcctgatcggaccgtcagtcttcctcttccccccaaaaccc

SEQ ID NO: 47 - DNA, 63 bp, homo sapiens
ccaccgtgcccagcacctgaactcctggggatcccgtcagtcttcctcttccccccaaaaccc

SEQ ID NO: 48 - DNA, 63 bp, homo sapiens
ccaccgtgcccagcacctgaacgcctggggggaccgtcagtcttcctcttccccccaaaaccc

SEQ ID NO: 49 - DNA, 63 bp, homo sapiens
ccaccgtgcccagcacctgaactccgcgggggaccgtcagtcttcctcttccccccaaaaccc

SEQ ID NO: 50 - DNA, 63 bp, homo sapiens
ccaccgtgcccagcacctgaactcctgcgcggaccgtcagtcttcctcttccccccaaaaccc

SEQ ID NO: 51 - DNA, 63 bp, homo sapiens
ccaccgtgcccagcacctgaactcctggggcgcccgtcagtcttcctcttccccccaaaaccc

SEQ ID NO: 52 - DNA, 63 bp, homo sapiens
ccaccgtgcccagcacctgaacccctggggggaccgtcagtcttcctcttccccccaaaaccc

SEQ ID NO: 53 - DNA, 63 bp, homo sapiens
ccaccgtgcccagcacctgaactccccgggggaccgtcagtcttcctcttccccccaaaaccc

SEQ ID NO: 54 - DNA, 63 bp, homo sapiens
ccaccgtgcccagcacctgaactcctgcccggaccgtcagtcttcctcttccccccaaaaccc

SEQ ID NO: 55 - DNA, 63 bp, homo sapiens
ccaccgtgcccagcacctgaactcctggggcccccgtcagtcttcctcttccccccaaaaccc

SEQ ID NO: 56 - DNA, 63 bp, homo sapiens
ccaccgtgcccagcacctgaatccctggggggaccgtcagtcttcctcttccccccaaaaccc

SEQ ID NO: 57 - DNA, 63 bp, homo sapiens
ccaccgtgcccagcacctgaactctccgggggaccgtcagtcttcctcttccccccaaaaccc

SEQ ID NO: 58 - DNA, 63 bp, homo sapiens
ccaccgtgcccagcacctgaactcctgtccggaccgtcagtcttcctcttccccccaaaaccc

SEQ ID NO: 59 - DNA, 63 bp, homo sapiens
ccaccgtgcccagcacctgaactcctggggtccccgtcagtcttcctcttccccccaaaaccc

SEQ ID NO: 60 - DNA, 63 bp, homo sapiens
ccaccgtgcccagcacctgaaggcctggggggaccgtcagtcttcctcttccccccaaaaccc

SEQ ID NO: 61 - DNA, 63 bp, homo sapiens
ccaccgtgcccagcacctgaactcggcgggggaccgtcagtcttcctcttccccccaaaaccc

SEQ ID NO: 62 - DNA, 63 bp, homo sapiens
ccaccgtgcccagcacctgaactcctggggggaggctcagtcttcctcttccccccaaaaccc

SEQ ID NO: 63 - DNA, 63 bp, homo sapiens
ccaccgtgcccagcacctgaactcctggggggaccgggcgtcttcctcttccccccaaaaccc

SEQ ID NO: 64 - DNA, 63 bp, homo sapiens
ccaccgtgcccagcacctgaactcctggggggaccgtcaggcttcctcttccccccaaaaccc

SEQ ID NO: 65 - DNA, 41 bp, homo sapiens
tgaactcctggggggaccgtgagtcttcctcttccccccaa

SEQ ID NO: 66 - DNA, 41 bp, homo sapiens
tggtggtggacgtgagccactaagaccctgaggtcaagttc

SEQ ID NO: 67 - DNA, 48 bp, homo sapiens
aatgccaagacaaagccgcgagaggagtagtacaacagcacgtaccgt

SEQ ID NO: 68 - DNA, 41 bp, homo sapiens
acaagtgcaaggtctccaactaagccctcccagcccccatc

SEQ ID NO: 69 - DNA, 63 bp, homo sapiens
ccaccgtgcccagcacctgaactcctggggggaccgtcagtcttcctcttccccccaaaaccc

SEQ ID NO: 70 - DNA, 63 bp, homo sapiens
gaggtcacatgcgtggtggtggacgtgagccacgaagaccctgaggtcaagttcaactggtac

SEQ ID NO: 71 - DNA, 63 bp, homo sapiens
aagacaaagccacgggaggagcagtacaacagcacgtaccgtgtggtcagcgtcctcaccgtc

SEQ ID NO: 72 - DNA, 63 bp, homo sapiens
tacaagtgcaaggtctccaacaaagccctcccagcccccatcgagaaaaccatttcgaaagcc

SEQ ID NO: 73 - protein, 245 aa, homo sapiens
MEGPRGWLVLCVLAISLASMVTEDLCRAPDGKKGEAGRPGRRGRPGLKGEQGEPGAPGIRTGIQGLKGDQGEPGPSGNPGKVGYPGPSGPLGARGIPGIKGTKGSPGNIKDQPRPAFSAIRRNPPMGGNVVIFDTVITNQEEPYQNHSGRFVCTVPGYYYFTFQVLSQWEICLSIVSSSRGQVRRSLGFCDTTNKGLFQVVSGGMVLQLQQGDQVWVEKDPKKGHIYQGSEADSVFSGFLIFPSA

SEQ ID NO: 74 - protein, 253 aa, homo sapiens
MMMKIPWGSIPVLILLLLLGLIDISQAQLSCTGPPAIPGIPGIPGTPGPDGQPGTPGIKGEKGLPGLAGDHGEFGEKGDPGIPGNPGKVGPKGPMGPKGGPGAPGAPGPKGESGDYKATQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNSLLGMEGANSIFSGFLLFPDMEA

SEQ ID NO: 75 - protein, 245 aa, homo sapiens
MDVGPSSLPHLGLKLLLLLLLLPLRGQANTGCYGIPGMPGLPGAPGKDGYDGLPGPKGEPGIPAIPGIRGPKGQKGEPGLPGHPGKNGPMGPPGMPGVPGPMGIPGEPGEEGRYKQKFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPD

SEQ ID NO: 76 - protein, 194 aa, homo sapiens
MGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQL

SEQ ID NO: 77 - protein, 194 aa, homo sapiens
MGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQL

SEQ ID NO: 78 - protein, 310 aa, homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 79 - DNA, 1470 bp, homo sapiens
ctgctgtgctctgggcgccagctcgctccagggagtgatgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaggaggactctgtgactctgacatgccgggggactcacagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagctctcccaggataccctgagtgcagggaaatgggagagaccctccctgagaaaccagccaatcccactaatcctgatgaggctgacaaagttggggctgagaacacaatcacctattcacttctcatgcacccggatgctctggaagagcctgatgaccagaaccgtatttagtctccattgtcttgcattgggatttgagaagaaaatcagagagggaagatctggtatttcctggcctaaattccccttggggaggacagggagatgctgcagttccaaaagagaaggtttcttccagagtcatctacctgagtcctgaagctccctgtcctgaaagccacagacaatatggtcccaaatgaccgactgcaccttctgtgcttcagctcttcttgacatcaaggctcttccgttccacatccacacagccaatccaattaatcaaaccactgttattaacagataatagcaacttgggaaatgcttatgttacaggttacgtgagaacaatcatgtaaatctatatgatttcagaaatgttaaaatagactaacctctaccagcacattaaaagtgattgtttctgggtgataaaattattgatgatttttattttctttatttttctataaagatcatatattacttttataataaaacattataaaaac

SEQ ID NO: 80 - protein, 60 aa, homo sapiens
MSTESMIRDVELAEEALPQKMGGFQNSRRCLCLSLFSFLLVAGATTLFCLLNFGVIGPQR

SEQ ID NO: 81 - protein, 60 aa, homo sapiens; identical to SEQ ID NO: 80

SEQ ID NO: 82 - protein, 40 aa, homo sapiens
RSSSQNSSDQPTHTCPPCPAPELLGGPSVFLFPPKPKDTL

SEQ ID NO: 83 - protein, 31 aa, homo sapiens
DEKFPNGLPLISSMAQTLTLRSSSQNSSDKP

SEQ ID NO: 84 - DNA, 100 bp, homo sapiens
atgagcacagaaagcatgatccgcgacgtggaactggcagaagaggcacttactcgtgtctttcgtactaggcgctgcaccttgaccgtcttctccgtga

SEQ ID NO: 85 - DNA, 100 bp, homo sapiens
cccccaaaagatggggggcttccagaactccaggcggtgcctatgtctcagggggttttctaccccccgaaggtcttgaggtccgccacggatacagagt

SEQ ID NO: 86 - DNA, 100 bp, homo sapiens
gcctcttctcattcctgcttgtggcaggggccaccacgctcttctgtctacggagaagagtaaggacgaacaccgtccccggtggtgcgagaagacagat

SEQ ID NO: 87 - DNA, 100 bp, homo sapiens
ctgaacttcggggtgatcggtccccaaagggatgagaagttcccaaatgggacttgaagccccactagccaggggtttccctactcttcaagggtttacc

SEQ ID NO: 88 - DNA, 100 bp, homo sapiens
catcagttctatggcccagaccagatcatcttctcaaaactcgagtgaccgtagtcaagataccgggtctggtctagtagaagagttttgagctcactgg

SEQ ID NO: 89 - DNA, 40 bp, homo sapiens
agccttgaggatccggatcctcggaactcctaggcctagg

SEQ ID NO: 90 - DNA, 990 bp, homo sapiens
atgagcacagaaagcatgatccgcgacgtggaactggcagaagaggcactcccccaaaagatggggggcttccagaactccaggcggtgcctatgtctcagcctcttctcattcctgcttgtggcaggggccaccacgctcttctgtctactgaacttcggggtgatcggtccccaaagggatgagaagttcccaaatggcatcagttctatggcccagaccagatcatcttctcaaaactcgagtgaccagccttatccatatgatgttccagattatgctactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaagaacaaaaactcatctcagaagaggatctgaat

SEQ ID NO: 91 - DNA, 543 bp, homo sapiens
gctgctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccgtttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatg

SEQ ID NO: 92 - protein, 184 aa, homo sapiens
AAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGII

SEQ ID NO: 93 - protein, 184 aa, homo sapiens
AAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSRLDPTFSIPQANHSHSGDYHCTGNIGYTLFSSKPVTITVQVPSMGSSSPMGII

SEQ ID NO: 94 - DNA, 40 bp, Artificial sequence
  synthetic
tatgatgttccagattatgctactcacacatgcccaccgt

SEQ ID NO: 95 - DNA, 40 bp, Artificial sequence
  synthetic
gcacggtgggcatgtgtgagtagcataatctggaacatca

SEQ ID NO: 96 - DNA, 21 bp, Artificial sequence
  synthetic
agtaacggccgccagtgtgct

SEQ ID NO: 97 - DNA, 39 bp, Artificial sequence
  synthetic
gcacggtgggcatgtgtgagtagcataatctggaacatc

SEQ ID NO: 98 - DNA, 39 bp, Artificial sequence
  synthetic
tccctgtccccgggtaaagaacaaaaactcatctcagaa

SEQ ID NO: 99 - DNA, 21 bp, Artificial sequence
  synthetic
agaaggcacagtcgaggctga

SEQ ID NO: 100 - DNA, 12 bp, homo sapiens
ctgctgggggga

SEQ ID NO: 101 - DNA, 54 bp, homo sapiens
  misc_feature (23)..(23): n is a, c, g, or t
ccaccgtgcccagcacctgaaandccgtcagtcttcctcttccccccaaaaccc

SEQ ID NO: 102 - DNA, 63 bp, homo sapiens; identical to SEQ ID NO: 26

SEQ ID NO: 103 - DNA, 21 bp, Artificial sequence
  synthetic
atatttacagaatggcacagg

SEQ ID NO: 104 - DNA, 20 bp, Artificial sequence
  synthetic
gacttggtacccaggttgaa

SEQ ID NO: 105 - DNA, 32 bp, Artificial sequence
  synthetic
atcagattcgatcctacttctgcagggggcat

SEQ ID NO: 106 - DNA, 32 bp, Artificial sequence
  synthetic
acgtgctgagcttgagtgatggtgatgttcac

SEQ ID NO: 107 - DNA, 22 bp, Artificial sequence
  synthetic
ggaaaatcccagaaattctcgc

SEQ ID NO: 108 - DNA, 27 bp, Artificial sequence
  synthetic
caacagcctgactacctattacgcggg

SEQ ID NO: 109 - DNA, 4 bp, Artificial sequence
  synthetic
cgcg

(19) United States
(12) Patent Application Publication
(10) Pub. No.: US 2009/0215639 A1
(43) Pub. Date: Aug. 27, 2009

(54) Method of Producing Human IgG Antibodies with Enhanced Effector Functions

(75) Inventors: Roberto Crea, Foster City, CA (US); Arvind Rajpal, San Francisco, CA (US); Guido Cappuccilli, San Mateo, CA (US); Toshihiko Takeuchi, Oakland, CA (US); Ramesh Bhatt, Belmont, CA (US); Randy Shen, Santa Clara, CA (US)

Correspondence Address: PHARMACIA CORPORATION;GLOBAL PATENT DEPARTMENT, POST OFFICE BOX 1027, ST. LOUIS, MO, 63006, US

(73) Assignee: Bioren, Inc., New York, NY (US)

(21) Appl. No.: 11/912,568

(22) PCT Filed: Apr. 26, 2006
(86) PCT No.: PCT/IB06/01030

§ 371 Date: Sep. 30, 2008

Related U.S. Application Data
(60) Provisional application No. 60/675,345, filed on Apr. 26, 2005.

(51) Int. Cl. C40B 30/04; C40B 30/00; C12P 19/34

(52) U.S. Cl. 506/9; 506/7; 435/91.5

(57) ABSTRACT

A method for generating human IgG₁ antibodies with enhanced Fc effector function is disclosed. In practicing the method, an IgG₁ Fc look-through mutagenesis (LTM) coding library directed at four receptor-contact regions of the Fc C_{H}2 portion of in human IgG₁ Fc is expressed in a system in which the mutated Fc fragments are displayed on the surfaces of the expression cells. The fragments are then screened for altered binding affinity to a selected Fc receptor or other Fc-binding protein. The selected mutations may be used, in turn, to guide the selection of multiple substitutions in the construction of a walk-through mutation (WTM) library, for generating additional Fc fragment mutations with desired binding properties. The antibodies so produced have a variety of therapeutic and diagnostic applications.

### FIELD OF THE INVENTION

The present invention relates to methods of producing human IgG antibodies, particularly IgG₁ antibodies, including fragment thereof, with enhanced effector functions.

### BACKGROUND OF THE INVENTION

Formation of an antibody-antigen complex and recognition by specialized immune cells triggers a wide range of immune system responses. The most common antibody isotype is IgG, composed of two identical heavy chains that are disulfide linked to two identical light chains. Antigen recognition occurs in the complementarity determining region formed at the terminal end of the associated heavy and light chains. At the other antibody terminus, interactions initiated through the binding of the antibody Fc domain to Fc receptors, leads to Fc effector functions such as antibody-dependent cell-mediated cytotoxicity (ADCC), cell-mediated complement activation (CDC), and phagocytosis (opsonization).

Fc receptors are cell-surface glycoproteins found on particular immune cells that can bind the terminal Fc portion of an antibody. These Fc receptors are defined by their distribution, immunoglobulin subtypes specificity and the effector response initiated. For example, FcγR receptors found on macrophages, peripheral blood mononuclear cells (PBMCs), and natural killer cells (NK) are more specific for IgG type molecules. NK cell FcRγIIIa receptor binding of the antibody bound target then mediates ADCC target cytolysis. Activation of the complement cascade on the other hand, is initiated by binding of serum complement protein C1q to the Fc portion of an antibody-antigen complex. Though not a cell surface molecule, C1q can still be considered an Fc receptor as C1q can direct either CDC or phagocytosis by recruiting deposition of the C3 complement component, followed by recognition by C3 receptors on various phagocytic cells.

Each human IgG heavy chain has an antigen recognizing variable domain (V) and 3 homologous constant-region domains; C_{H}1, C_{H}2 and C_{H}3 where the C_{H}2 and C_{H}3 comprise the Fc region. Mutagenesis studies have shown that it is the C_{H}2 and C_{H}3 domains that most important to these Fc receptor mediated responses. Thus, by identifying the key Fc amino acid residues mediating Fc receptor interactions, antibody Fc engineering could potentially provide new capabilities and improvements to selectively increase Fc-effector functions, alter FcR targeting for more efficient radionuclide or cytotoxic drug targeting and/or optimize therapeutic half life modalities requiring chronic dosing regimens.

It would thus be desirable to provide a systematic mutagenesis and screening method by which beneficial mutations throughout the entire Fc region for chosen Fc effector properties can be rapidly and efficiently identified. To facilitate the screening method, it would be further desirable to provide a method in which Fc mutations are expressed as a mammalian Fc variant library on the surface of mammalian cells, such that the Fc variants can be directly screened by in vitro ADCC and/or CDC assay readouts.

### SUMMARY OF THE INVENTION

The invention includes, in one aspect, a method of generating human IgG, antibodies with enhanced effector function. In carrying out the method, there is constructed an IgG₁ Fc look-through mutagenesis (LTM) coding library. The library may be a regional LTM library encoding, for at least one of the two IgG₁ Fc regions identified by SEQ ID NOS: 1 and 2, representing the C_{H}2 and C_{H}3 regions of the antibody's Fc fragment, respectively, and for each of a plurality of amino acids, individual amino acid substitutions at multiple amino acid positions within one of the two IgG₁ Fc regions. Alternatively, the library may be a sub-region LTM library encoding, for each of the four regions identified by SEQ ID NOS: 14-17 contained within the IgG₁ Fc C_{H}2 region identified by SEQ ID NO:1, and for each of a plurality of selected amino acids, individual substitutions at multiple amino acid positions within each region.

The IgG₁ Fc fragments encoded by the LTM library are expressed in a selectable expression system, and those expressed IgG₁ Fc fragments that are characterized by an enhanced effector function are selected. The enhanced effector function is related to (i) a shift in binding affinity constant (K_{D}), with respect to a selected IgG₁ Fc binding protein, relative to native IgG₁ Fc; or (ii) a shift in the binding off-rate constant (K_{off}); with respect to a selected IgG₁ Fc binding protein, relative to native IgG₁ Fc, and may be based on either a direct K_{D} or K_{off} measurement or an indirect measure of binding, such as antibody-dependent cell-mediated cytotoxicity (ADCC), cell-mediated complement activation (CDC), and phagocytosis (opsonization).

The expressed Fc fragments encoded by the library may be expressed in a selectable expression system composed of viral particles, prokaryotic cells, and eukaryotic cells, where the expressed Fc particles are attached to the surface of the expression-system particles and accessible thereon to binding by the Fc binding protein. One exemplary expression system includes a mammalian cell, such as a BaF3, FDCP1, CHO, and NS0 cell, that is (i) capable of producing clinical-grade monoclonal antibodies, (ii) nonadherent in culture, and (iii) readily transduced with a retrovirus.

The expression system may include a mammalian cell that expresses the Fc fragments on its surface, allowing a direct measure of Fc effector function, such as antibody-dependent cell-mediated cytotoxicity (ADCC), cell-mediated complement activation (CDC), and phagocytosis (opsonization). This direct method includes the steps of (i) adding expression cells corresponding to a single clonal variant of the LTM library to each of a plurality of assay wells, (ii) adding to each well, reagents that include an Fc binding protein and which are effective to interact with the surface-attached Fc fragment, and depending on the level of binding thereto, to lyse the cells, (iii) assaying the contents of the wells for the presence of cell lysis products, and (iv) selecting those IgG₁ Fc fragments which are expressed on cells showing the greatest level of cell lysis.

For measuring ADCC directly, the reagents added in step (ii) may be peripheral blood mononuclear cells capable of lysing cells expressing the Fc fragment on their surface by antibody-dependent cellular cytotoxicity. The method may further include, prior to step (i), enriching such cells for those expressing Fc fragments having an elevated binding affinity constant or reduced binding off-rate constant, with respect to Fc-binding proteins FcγRI or FcγRIIIa.

For measuring CDC directly, the reagents added in step (ii) are human C1q complex and human serum, capable of lysing cells by complement-mediated cell death. The method may further include, prior to step (i), enriching such cells for those expressing Fc fragments having an elevated binding affinity constant or reduced binding off-rate constant, with respect to Fc-binding protein C1q.

In both cases of direct measuring of effector function, the method may further include enriching the cells for those expressing Fc fragments having one of: (i) an elevated binding affinity constant or reduced binding off-rate constant, with respect to Fc-binding protein C1q, FcγRI, FcγRIIa, and FcγRIIIa, (ii) a reduced binding affinity constant or elevated binding off-rate constant with respect to Fc-binding proteins FcγRIIb, FcγRIIIb; and (iii) an elevated or reduced binding affinity constant or a reduced or elevated binding off-rate constant, respectively, with respect to Fc-binding protein FcRN and protein A.

For generating expressed Fc fragments having an elevated binding affinity constant, with respect to Fc-binding protein selected from the group consisting of C1q, FcγRI, FcγRIIa, FcγRIIIa, FcRN and protein, relative to the binding affinity constant for native IgG₁ Fc fragment, the selecting step may include (i) forming a mixture of expression particles with displayed Fc fragments and an Fc binding protein, (ii) allowing the Fc binding protein to bind with the displayed Fc fragments in the mixture, to form an Fc-binding complex, and (iii) isolating the Fc-binding complexes from the mixture, wherein particles expressing Fc fragments having the highest binding affinity constants for the binding protein are isolated.

For generating Fc fragments having an elevated equilibrium binding affinity constant, with respect to Fc-binding protein selected from the group consisting of C1q, FcγRI, FcγRIIa, FcγRIIIa, FcRN and protein A, relative to the binding affinity constant for native IgG₁ Fc fragment, the selecting may step include (i) forming a mixture of expression particles with displayed Fc fragments and a limiting amount of fluorescent-labeled Fc binding protein in soluble form, such that those particles expressing Fc fragments with a higher binding affinity constant will be more strongly labeled, (ii) after the binding in the mixtures reaches equilibrium, sorting the particles on the basis of amount of bound fluorescent label, and (iii), selecting those particles having the highest levels of bound fluorescence.

For generating Fc fragments having a reduced binding off-rate constant, with respect to Fc-binding protein selected from the group consisting of FcγRIIb, FcγRIIIb, FcRN and protein A, relative to the binding affinity constant for native IgG₁ Fc fragment, the selecting step may include (i) forming a mixture of expression particles with displayed Fc fragments and a limiting amount of fluorescent-labeled Fc binding protein in soluble form, such that those particles expressing Fc fragments with a lower binding affinity constant will be less strongly labeled, (ii) after the binding in the mixtures reaches equilibrium, sort the particles on the basis of amount of bound fluorescent label, and (iii), selecting those particles having the lowest levels of bound fluorescence.

For generating Fc fragments having a reduced binding off-rate affinity constant, with respect to Fc-binding protein selected from the group consisting of C1q, FcγRI, FcγRIIa, FcγRIIIa, FcRN and protein A, relative to the binding affinity constant for native IgG₁ Fc fragment, the selecting step may include (i) forming a mixture of expression particles with displayed Fc fragments and a saturating amount of fluorescent-labeled Fc binding protein in soluble form, (ii) at a selected time after step (i), adding a saturating amount of an unlabeled Fc binding protein, (iii) at a selected time after step (ii) and prior to binding equilibrium, sort the particles on the basis of amount of bound fluorescent label, and (iv), selecting those particles having the highest levels of bound fluorescence.

For generating Fc fragments having an increased binding off-rate affinity constant, with respect to Fc-binding protein selected from the group consisting of FcγRIIb, FcγRIIIb, FcRN and protein A, relative to the binding affinity constant for native IgG₁ Fc fragment, the method may include (i) forming a mixture of expression particles with displayed Fc fragments and a saturating amount of fluorescent-labeled Fc binding protein in soluble form, (ii) at a selected time after step (i), adding a saturating amount of an unlabeled Fc binding protein, (iii) at a selected time after step (cii) and prior to binding equilibrium, sort the particles on the basis of amount of bound fluorescent label, and (iv), selecting those particles having the lowest levels of bound fluorescence.

For generating Fc fragments having the ability, when incorporated into an IgG, antibody, to enhance antibody-dependent cellular-toxicity, the method may further include, after identifying IgG₁ Fc fragments characterized by an elevated binding affinity constant or reduced binding off-rate constant for FcγRIIIA, the selecting step may further include selecting the identified fragments for binding affinity for the FcγRIIB receptor that exhibits reduced binding affinity constant or elevated binding off-rate constant for the FcγRIIB receptor.

For generating Fc fragments having the ability, when incorporated into an IgG₁ antibody, to enhance complement-dependent cytotoxicity (CDC), wherein step (c) further includes, after identifying IgG₁ Fc fragments characterized by an elevated binding affinity constant or reduced binding off-rate constant for C1q complex, the selecting step may further include selecting the identified fragments for binding affinity for the FcγRIIB receptor that exhibits reduced binding affinity constant or elevated binding off-rate constant for the FcγRIIB receptor.

For generating Fc fragments having the ability, when incorporated into an exogenous therapeutic IgG₁ antibody, to enhance the therapeutic response to the antibody in human patients having a position position-158 receptor polymorphism in the FcγRIIIA receptor, the selecting step may include selecting those expressed IgG₁ Fc fragments that are characterized by a binding affinity for the FcγRIIIA F158 receptor polymorphism that is at least as great as that for a FcγRIIIA V158 receptor polymorphism.

For generating Fc fragments having the ability, when incorporated into an exogenous therapeutic IgG₁ antibody, to enhance the therapeutic response to the antibody in human patients having a position-134 receptor polymorphism in the FcγRIIA receptor, the selecting step may include selecting those expressed IgG₁ Fc fragments that are characterized by a binding affinity for the FcγRIIA R131 receptor polymorphism that is at least as great as that for a FcγRIIA H131 receptor polymorphism.

The method may further include, after the initial selecting step, the steps of constructing a walk-through mutagenesis (WTM) library encoding, for at least one of the Fc coding regions at which amino acid substitutions are made in the LTM library, the same amino acid substitution at multiple amino acid positions within that region, where the substituted amino acid corresponds to an amino acid variation found in at least one amino acid position of an Fc fragment initially selected; expressing the IgG₁ Fc fragments encoded by the WTM library in a selectable expression system; and selecting those IgG₁ Fc fragments so expressed that are characterized by a desired shift in binding affinity constant or binding off-rate constant with respect to a selected IgG₁ Fc binding protein, compared with the same constant measured for a native Fc fragment.

The IgG₁ Fc fragments generated in the method may be characterized by an increased binding affinity constant or reduced binding off-rate constant for a human IgG₁ Fc-binding protein, where the shift in constant relative to the same constant measured for a native Fc fragment is greater than a factor of 1.5

The IgG₁ Fc fragments generated in the method may be characterized by an decreased binding affinity constant or increased binding off-rate constant for a human IgG₁ Fc-binding protein, where the shift in constant relative to the same constant measured for a native Fc fragment is greater than a factor of 1.5

These and other objects and features of the invention will become more fully apparent when the following detailed description of the invention is read in conjunction with the accompanying drawings.

### BRIEF DESCRIPTION OF THE DRAWINGS

FIGS. 1A-1C illustrate a schematic structure of an IgG₁ antibody (1A), showing the Fc portion-pointing out the C_{H}2 and C_{H}3 regions thereof, (1B) the recruitment of the complement component C1q by binding to the C_{H}2 of the antibody for CDC function, and (1C) the recruitment of the FcγRIIIa by binding the C_{H}2 fragment for ADCC function.

FIG. 2 illustrates the C_{H}2 and C_{H}3 regions of the “unbiased” Fc effector library. See SEQ ID: 12 and 13 for the delineated sections. The calculation below is the predicted number of possible LTM variants in creating the C_{H}2 and C_{H}3 library combinations with the nine pre-selected LTM amino acids.

FIG. 3 shows a schematic array of LTM library combinations for both the C_{H}2 and C_{H}3 “unbiased” domains. For example, a possible “double” Fc-LTM library could consist of an Asp LTM library in C_{H}2 sub-region 8 followed by a H is Fc-LTM in C_{H}3 sub-region 1.

FIG. 4 shows the four “contact” sub-regions of the Fc C_{H}2 as identified from Fc domain-FcγRIIIa co-crystal structure. The smaller inset picture depicts the three-dimensional structure of human IgG Fc region highlighting (light/yellow) the amino acids in the four FcγRIIIa “contact” sub-regions. The calculation below is the predicted number of possible LTM variants in replacing the contact residues with the LTM amino acids and creating combinatorial multiple LTM replacement libraries between “contact” sub-regions.

FIG. 5 illustrates the nine LTM amino acid substitutions at each regional position of the first “contact” sub-region of the Fc C_{H}2 domain, in accordance with the LTM selection method employed in the present invention.

FIG. 6 shows the 4 oligonucleotide coding sequences corresponding to the asparagine substitution polypeptides shown in FIG. 5.

FIG. 7 shows all the possible Fc-LTM library combinations in the CH2 domain for analysis of the four Fc-FcγRIIIa “contact” sub-regions. Each “contact” sub-region LTM library is comprised of the single amino acid replacements by the nine pre-selected LTM amino acids in each and every position in the “contact” sub-region. For example, a possible “triple” Fc-LTM library could consist of an Arg LTM library in “contact” sub-region 1, have NO LTM analysis in “contact” sub-region 2 followed by a Pro Fc-LTM in “contact” sub-region 3 and H is Fc-LTM in “contact” sub-region 4.

FIG. 8 is an illustrative example of a degenerate oligonucleotide for combinatorial beneficial mutation analysis (CBM). The wild type amino acid and coding DNA sequence for Fc receptor “contact” sub-region 2 is shown in the upper portion. Hypothetical examples of Fc-LTM effector enhancing amino acid substitutions are in the diagram below. These Fc-LTM substitutions are indicated above the wild type amino acid. For CBM (see Example 11) the necessary nucleotides at each codon for incorporating the desired changes in various combinations are then shown in the degenerate oligonucleotide below.

FIG. 9 shows various schematic representative IgG1 and Fc-fragment chimeric molecules that are formed in accordance with the present invention. The top four chimeric constructs are comprised of a N-terminal leader sequence for extracellular export, the Fc domain, and a C-terminal membrane anchoring signal to retain the protein. The bottom chimeric construct illustrates an example of a Type II N-terminal anchor whereby the modified TNF-α leader is both a extracellular secretion and transmembrane anchor signal.

FIGS. 10A and 10B illustrate a C-terminal (10A) and a Type II N-terminal Anchored Fc display system (10B). The Type II N-terminal display system illustrates that CH3 distal orientation is more biological similar to the natural presentation of an IgG₁ bound to the target antigen on a cell.

FIG. 11 shows the pDisplay expression vector for cloning the Fc-LTM construct in between the N-terminal IgK leader and C-terminal PDGF receptor transmembrane anchor.

FIG. 12 shows the schematic design of a vector utilizing Type II N-terminal anchor from the TNF extracellular leader and the Fc-LTM construct for cell surface display.

FIGS. 13A and 13B illustrate illustrate the Kunkel mutageneisis method as applied in the present invention for generating Fc coding sequences using a single oligonucletide annealing reaction (FIG. 13A) and multiple oligonucleotide (FIG. 13B), the first modified Fc-LTM template must be re-isolated and re-annealed with a second different oligonucleotide to generate two separately located Fc-LTM mutations. These iterations are then repeated until the desired Fc-LTM mutations are incorporated.

FIGS. 14A and 14B show the results of oligonucleotide annealing to replace the stop codon on the Fc mutagenesis template. In the Fc-LTM oligonucleotide annealed template (14A), a full length Fc-LTM protein is translated with a linked transmembrane signal allows cell surface retention. Translation of a truncated Fc-LTM protein also results in extracellular transport but, as there is no cell surface anchoring protein (indicated by the spotted oval), this chimeric Fc-LTM is then free to dissociate from the cell (FIG. 14B).

FIG. 15 shows the procedural steps of a transient retroviral expression system in accordance to the present invention. After transient transient transfection with the pDisplay Fc-LTM vectors, the pEco cell culture supernatant is harvested to collect pDisplay Fc-LTM retroviruses. The retroviruses then infect the library target cells of choice and individual clones are screened for desired properties. The clones are isolated and the Fc-LTM gene of interest is then recovered by PCR using conserved flanking primers for subsequent sequence analysis.

FIG. 16 shows a BIAcore sensorgram determination of binding kinetics of approximated varying concentrations of FcγRIIIa binding to immobilized IgG₁.

FIG. 17 illustrates the general steps and cellular binding components in the magnetic pre-selection of IgG₁ Fc fragments formed in accordance with the present invention for high binding affinity based on equilibrium binding to FcγRIIIa receptor.

FIG. 18 shows steps in the method for pre-selecting Fc fragments for high affinity binding to FcγRIIIa receptors in accordance with the invention;

FIG. 19 illustrates the flow diagram in the screening steps of IgG₁ Fc-LTM fragments formed in accordance with the present invention for high binding affinity based on equilibrium binding to a fluorescent-labeled FcγR receptors, i.e., FACS sorting for Fc clones based on equilibrium binding. Also shown is the optional step of the concurrent screening of Fc-LTM subpopulation which demonstrates lower FcγRIIb affinity.

FIGS. 20A and 20B are FACS plots showing a selection gate (the P2 trapezoid) for identifying those clones that express the cell surface protein of interest with enhanced binding affinity to a labeled associating protein. After equilibrium binding, the FACS profile will order clones with higher affinity by virtue of their higher fluorescent signal (Y-axis). A distribution of binding affinities is observed in the pre-sort population (A) and the higher affinity clones only comprise 6% of the total population. The post-sort (B) shows that there is greater than 25% of the sort population now display the desired enhanced binding affinity.

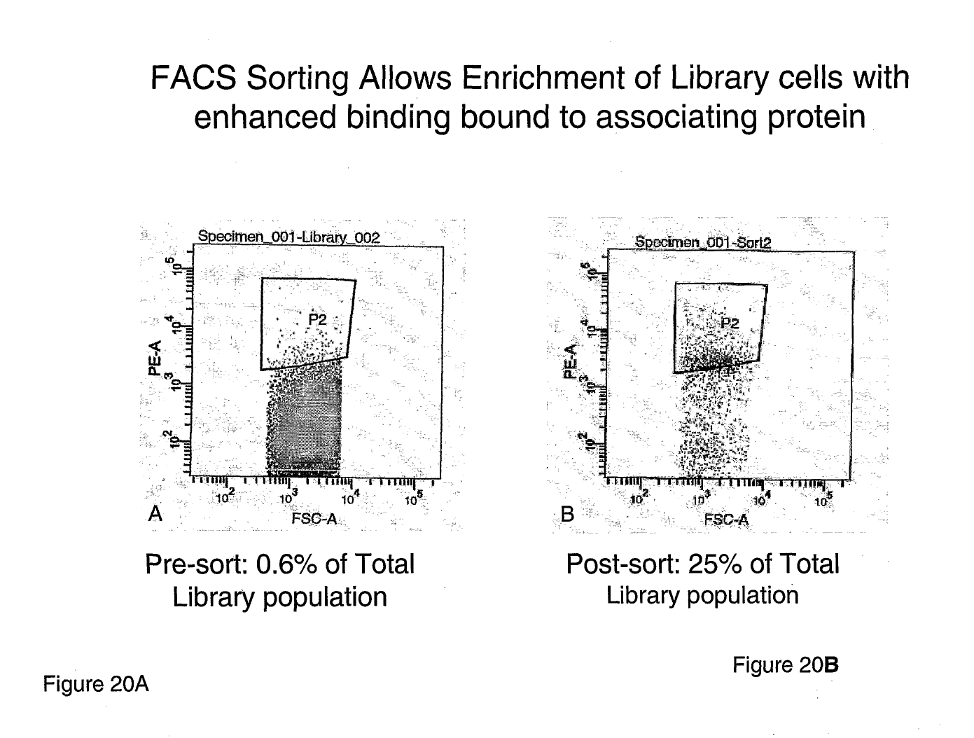

### DETAILED DESCRIPTION OF THE INVENTION

### I. Definitions

The terms below have the following definitions herein unless indicated otherwise.

The numbering of the residues in an IgG Fc fragment and the heavy chain containing the fragment is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG₁ EU antibody.

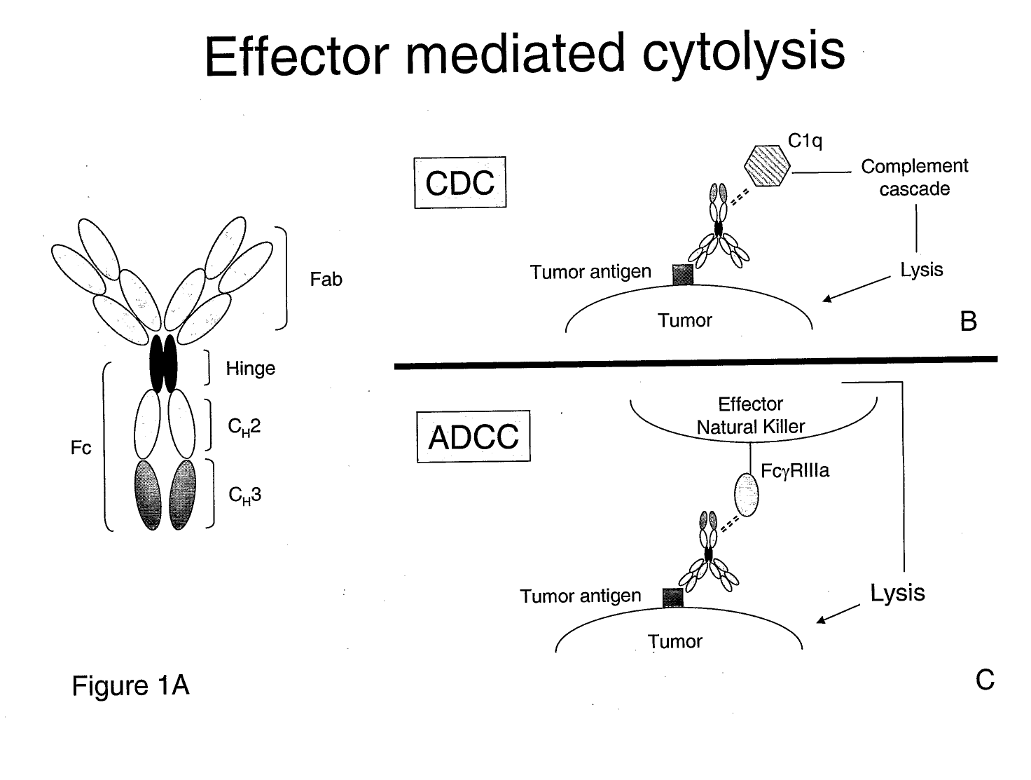

The term “Fc region” or “Fc fragment” is used to define a C-terminal region of an IgG heavy chain as shown in FIG. 1. The human IgG₁ Fc region is usually defined to stretch from amino acid residue at position Cys 226 to the carboxyl-terminus. The term “Fc region-containing polypeptide” refers to a polypeptide, such as an antibody or immunoadhesin (see definitions below), which comprises an Fc region. The term “Fc fragment” refers to the Fc region of an antibody ofr subregions thereof, e.g., the C_{H}2 or C_{H}3 region containing effector functions.

The Fc region of an IgG comprises two constant domains, C_{H}2 and C_{H}3, as shown in FIG. 1A. The “C_{H}2” domain of a human IgG Fc region (also referred to as “Cy2” domain) usually extends from amino acid 231 to amino acid 340. The C_{H}2 domain is unique in that it is not closely paired with another domain. Rather, two N-linked branched carbohydrate chains are interposed between the two, CH2 domains of an intact native IgG molecule.

“Hinge region” is generally defined as stretching from Glu216 to Pro230 of human IgG₁ (Burton, Molec. Immunol.22:161-206 (1985)) Hinge regions of other IgG isotypes may be aligned with the IgG₁ sequence by placing the first and last cysteine residues forming inter-heavy chain S—S bonds in the same positions.

“C1q” is a polypeptide that includes a binding site for the Fc region of an immunoglobulin. C1q together with two serine proteases, C1r and C1s, forms the complex C1, the first component of the complement dependent cytotoxicity (CDC) pathway. Human C1q can be purchased commercially from, e.g. Quidel, San Diego, Calif.

The term “Fc receptor” or “FcR” is used to describe a receptor that binds to the Fc region of an antibody. The preferred FcR is one, which binds an IgG antibody (a γ receptor) and includes receptors of the FcγRI, FcγRII, and FcγRIII subclasses, including allelic variants and alternatively spliced forms of these receptors. FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)). The term also include other polypeptides known to binding specifically to the Fc region of an IgG antibody, such as the C1q peptide complex and protein A.

The term “binding domain” refers to the region of a polypeptide that binds to another molecule. In the case of an FcR, the binding domain can comprise a portion of a polypeptide chain thereof (e.g. the α chain thereof) which is responsible for binding an Fc region. One useful binding domain is the extracellular domain of an FcR α chain.

The term “antibody” is used in the broadest sense and specifically covers monoclonal antibodies (including full length monoclonal antibodies), polyclonal antibodies, multispecific antibodies (e.g., bi-specific antibodies), and antibody fragments so long as they exhibit the desired biological activity.

The term “monoclonal antibody” as used herein refers to an antibody obtained from a population of substantially homogeneous antibodies, i.e., the individual antibodies comprising the population are identical except for possible naturally occurring mutations that may be present in minor amounts.

The term “K_{off}”, as used herein, is intended to refer to the off rate constant for dissociation of an antibody from the antibody/antigen complex, as determined from a kinetic selection set up. The units of a K_{off} rate constant is sec-1, indicating the rate of dissociation of a binding complex. A higher-valued K_{off} constant means a higher rate of dissociation and therefore a lower affinity between the two binding species. That is, the affinity between two binding species can be increased by reducing its K_{off}, and/or increasing its Kₒₙ.

The term “K_{D}”, as used herein, refers to the dissociation constant of a particular antibody-antigen interaction, and describes the concentration of antigen (expressed in M) required to occupy one half of all of the antibody-binding sites present in a solution of antibody molecules at equilibrium, and is equal to K_{off}/Kₒₙ, the on and off rate constants for the antibody. The association constant K_{A} of the antibody is 1/K_{D}. The measurement of K_{D} presupposes that all binding agents are in solution. In the case where the antibody is tethered to a cell wall, e.g., in a mammalian-cell expression system, the corresponding equilibrium rate constant is expressed as EC₅₀, which gives a good approximation of K_{D}. A lower the value of K_{D}, the higher the binding constant, i.e., a K_{D} of 10⁻⁸ M is greater affinity than 10⁻⁷ M.

The three-letter and one-letter amino acid abbreviations and the single-letter nucleotide base abbreviations used herein are according to established convention.

### II. Fc-LTM Libraries

This section describes Fc-LTM libraries employed in the method of the invention. As will be discussed more fully in Section IV below, the purpose of the libraries is to generate selected amino-acid substitution mutations in each or substantially each amino-acid position in one or more selected regions of the Fc fragment, to generate libraries of Fc fragments that can be screened for Fc fragments having enhanced effector function.

The Fc portion or fragment of an IgG antibody 20 are shown in FIG. 1A, and include 2 homologous constant-region domains 22, 24 referred to C_{H}2 and C_{H}3, which are known to be the domains that are most important to Fc receptor mediated responses. The “unbiased” LTM libraries will be localized within one or both of these domains; the “active-region” LTM library are typically localized in one-four regions of the C_{H}2 domain that are involved in Fc interactions with Fc receptor proteins.

Two important effector functions for which enhanced Fc function will be screened are cell-mediated cytotoxicity (CDC), and antibody-dependent cellular cytotoxicity ADCC), illustrated in FIGS. 1B and 1C, respectively, and considered further in Section IV below. In these and other applications, enhanced Fc effector functions will be related to (i) a shift in binding affinity constant (K_{D}), with respect to a selected IgG₁ Fc binding protein, relative to native IgG₁ Fc; and/or (ii) a shift in the binding off-rate constant (K_{off}); with respect to a selected IgG₁ Fc binding protein, relative to native IgG₁ Fc. As will be seen in Section IV, the Fc libraries can be screened directly for a change in binding constant, which can be an increase or decrease in binding constant, depending on the binding constant being measured, the Fc binding protein involved, and the desired effect of the change in binding constant. Alternatively, an enhancement in effector function can be measured directly, e.g., an increase or decrease in CDC or ADCC.

The LTM libraries and screening methods detailed below are applied specifically to generating enhanced Fc characteristics in IgG₁ type antibodies. However, it ill be appreciated that the methods can be applied as well to IgG₂, IgG₃, and IgG₄ subtypes of IgG antibodies, and Section B below discusses-various types of enhanced effector function that may be desired with each IgG subtype.

### A. FC-LTM Libraries

The purpose of look-through mutagenesis (LTM) is to introduce a selected substitution at each of a multiplicity of target mutation positions in a region of a polypeptide. Unlike combinatorial methods or walk-through mutagenesis (WTM), which allow for residue substitutions at each and every position in a single polypeptide (see below), LTM confines substitutions to a single selected position, i.e., a single substitution within a defined region or subregion.

The present invention contemplates two general types of Fc libraries constructed for L™ analysis, both of which are referred to below as an FC-LTM library. The first library is termed an “unbiased” C_{H}2×C_{H}3 library where each library coding sequence includes an amino acid substitution at one selected residue positions in the C_{H}2 region, and a single amino acid at one selected residue position in the C_{H}3 region, where the library preferably includes, at each or substantially each position in both regions, substitutions for each of a subset of chosen LTM amino acids, which collectively represent the major amino acid classes. That is, rather than examine the effect of all 20 natural L-amino acids; it is more efficient to employ a subset of these that represent the chemical diversity of the entire group. One representative subset of L-amino acids that meets this criterion includes the nine amino acids alanine, aspartate, lysine, leucine, proline, glutamine, serine, tyrosine, and histidine. These amino acids display adequate chemical diversity in size, charge, hydrophobicity, and hydrogen bonding ability to provide meaningful initial information on the chemical functionality needed to improve antibody properties.

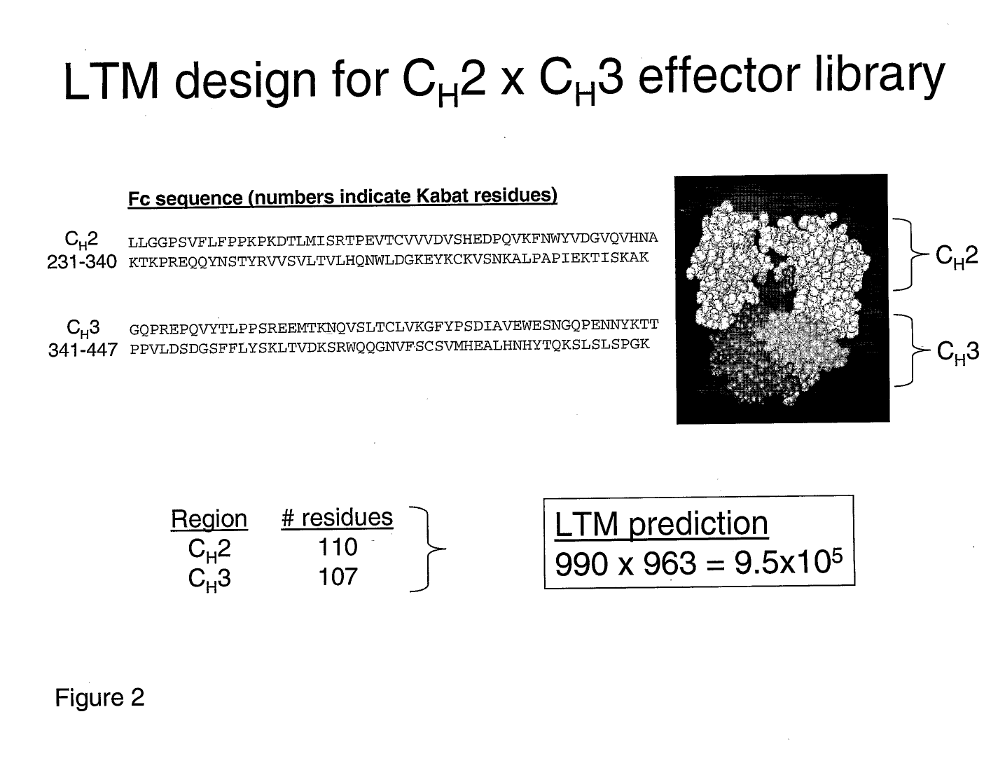

As seen in FIG. 2, there are 1926 LTM oligonucleotides (217 Fc domain amino acids×9 LTM amino acid replacements per Fc position) and are on average, 63 base pairs in length. For the “unbiased” Fc domain library, the CH2 (SEQ ID:1) and CH3 (SEQ ID:2) regions are artificially divided into juxtaposed subsections of 5 to 7 amino acid length (SEQ IDs:12 and 13 respectively). The 18 C_{H}2 and 16 C_{H}3 subsections thus individually represent portions of the contiguous full length IgG₁ Fc sequence. By placing one of nine different amino acids at one position in each of the C_{H}2 and C_{H}3 domains, one would generate 990×963 different library genes, or 9.5×10⁵ different library genes.

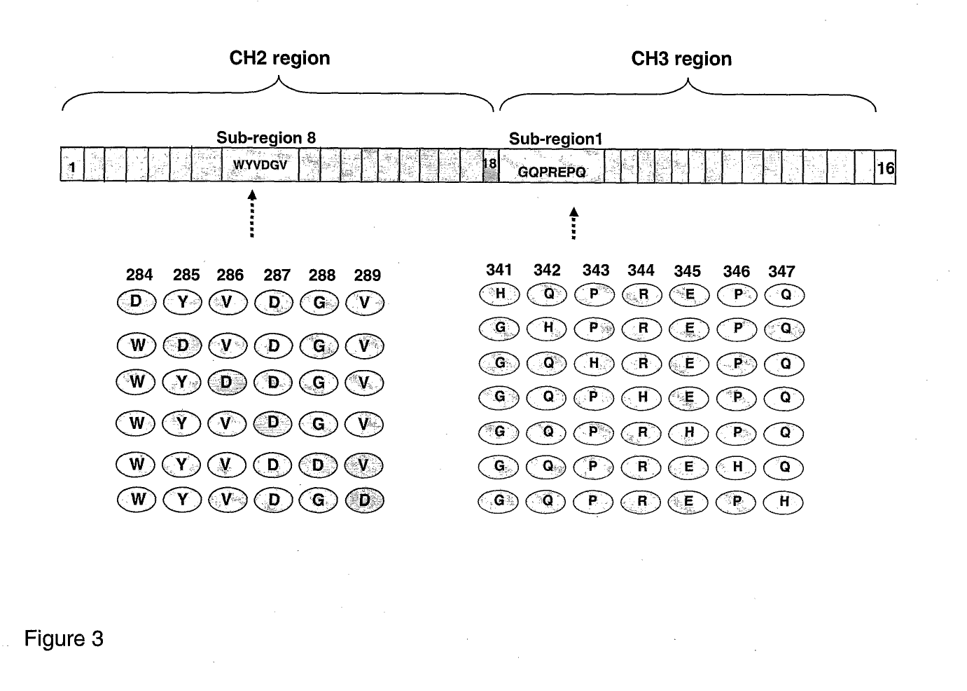

An alternative scheme for preparing an unbiased library containing a single mutation of one of, e.g., nine amino acids, at one position in each of the C_{H}2 and C_{H}3 domains is illustrated in FIG. 3. The figure shows one of 18 (arbitrary) subregions of the C_{H}2 region, and one of 16 subregions of the C_{H}3 region. The approach here is to produce 18×16 “unbiased” sublibraries for each of the 18 subregions in C_{H}2 and each of the 16 subregions in C_{H}3, where each of these sublibraries contains one of nine amino acid mutations at one position in a selected subregion, e.g., subregion-8 in C_{H}2 and at one position in selected subregion, e.g., subregion 1, of C_{H}3.

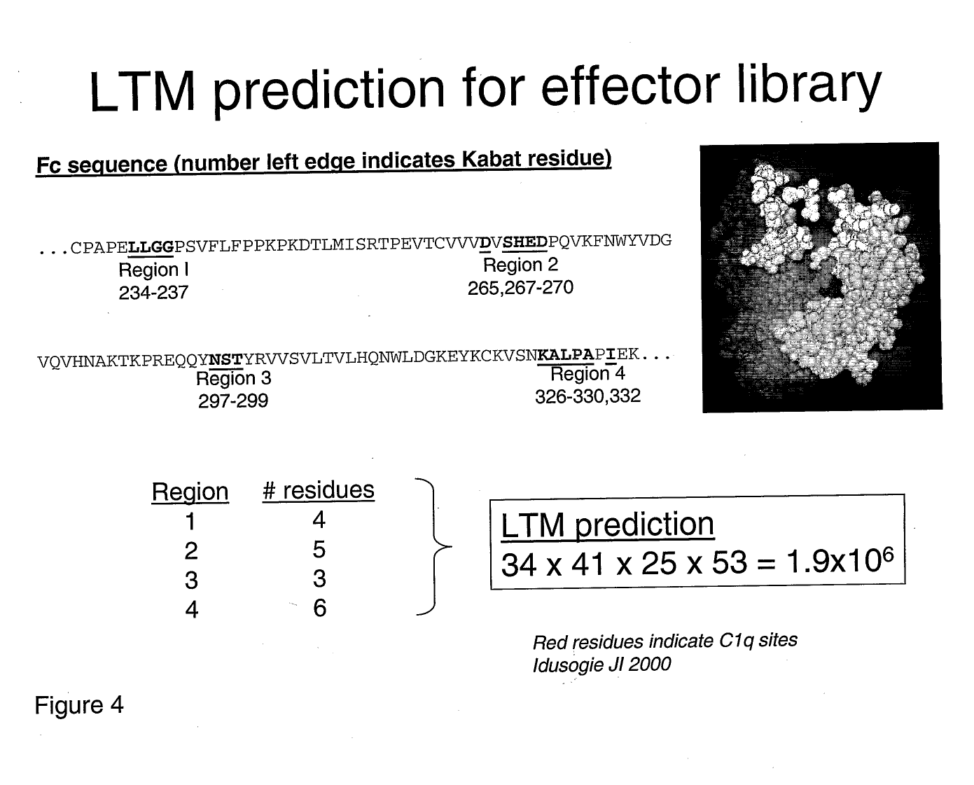

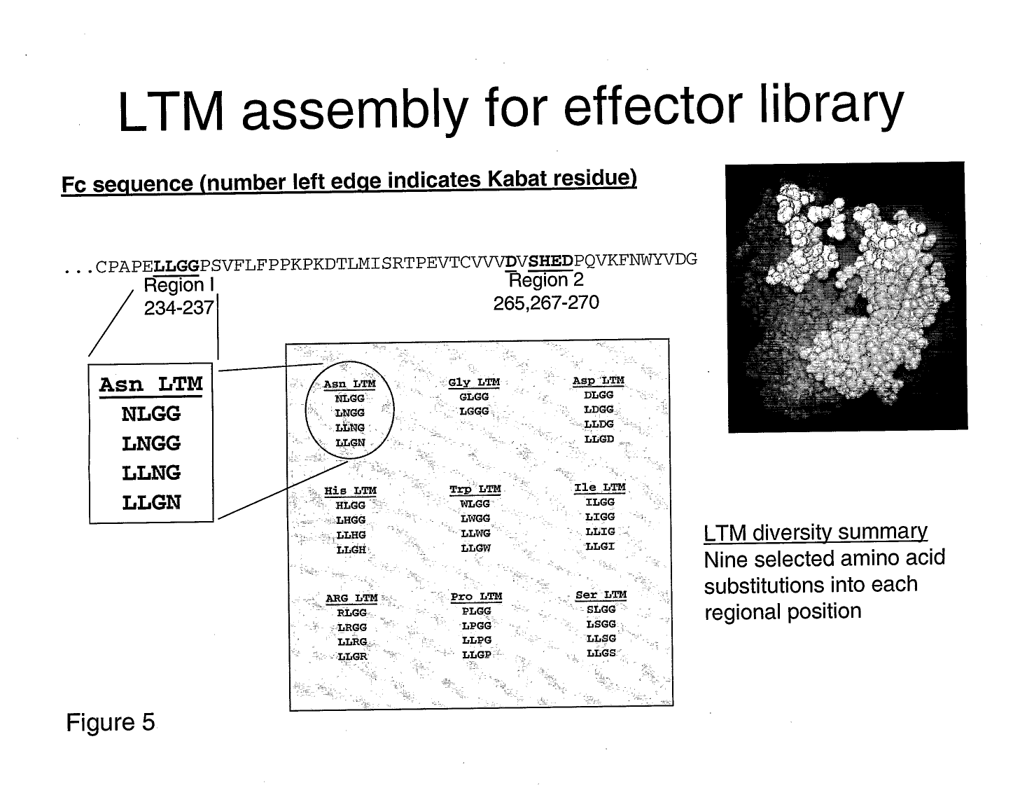

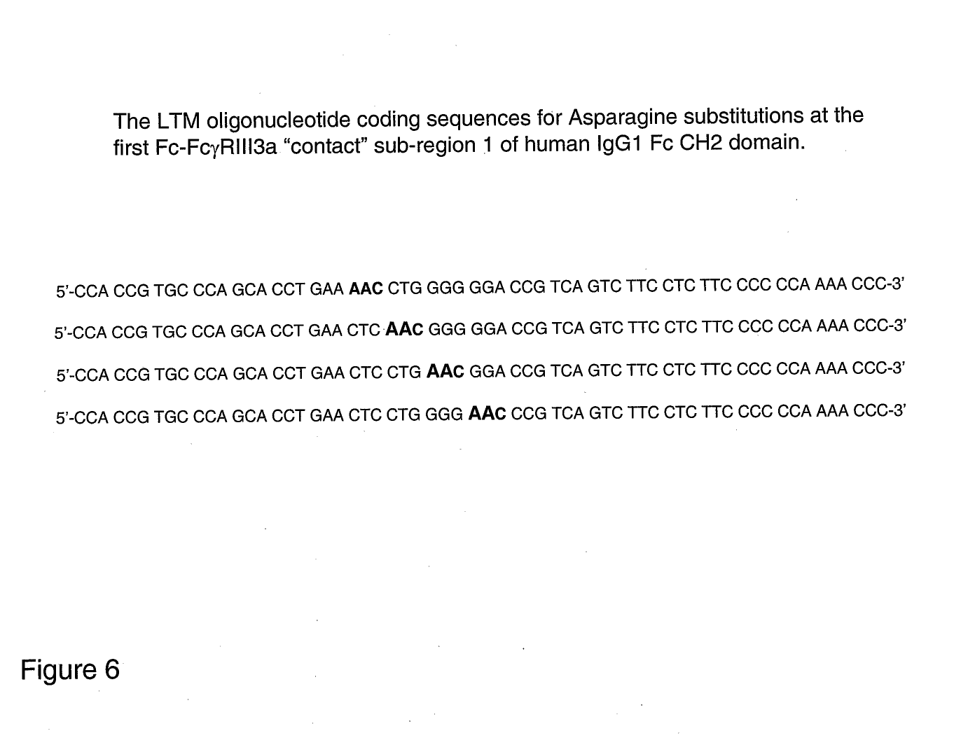

The second general type of Fc LTM library represents mutations at positions in one or more of four separate IgG₁ Fc-FcγRIIIa “contact” points as identified from the IgG1 Fc-FcγRIIIa co-crystal structure (FIG. 4). This second library then delineates four sub-regions (SEQ ID:14-17) within the total “unbiased” C_{H}2×C_{H}3 library above. The desired amino acid replacements at “contact” sub-region 1 are shown in FIG. 5. The “contact” sub-region 1: LLGG (SEQ ID:14) is coded for by the DNA sequence: CTG CTG GGG GGA and flanked by the DNA sequences 5′-cca ccg tgc cca gca cct gaa and ccg tca gtc ttc ctc ttc ccc cca aaa ccc-3′ framework. The four glycine LTM replacement oligonucleotides for “contact” sub-region 1 are listed (SEQ ID:18). The LTM oligonucleotide sequence: 5′-cca ccg tgc cca gca cct gaa GGG CTG GGG GGA ccg tca gtc ttc ctc ttc ccc cca aaa ccc-3′ demonstrates the glycine replacement codon (in bold). For “contact” sub-region 1, the remaining corresponding LTM oligonucleotides for asparagine (SEQ ID: 19), aspartate (SEQ ID: 20), histidine (SEQ ID: 21), tryptophan (SEQ ID: 22), iso-leucine (SEQ ID: 23), arginine (SEQ ID: 24), proline (SEQ ID: 25), and serine (SEQ ID: 26) show similar sequence design strategy. FIG. 6 illustrates the 4 LTM oligonucleotides for asparagines substitutions at the first contact subregion of IgG₁ Fc CH2 domain.

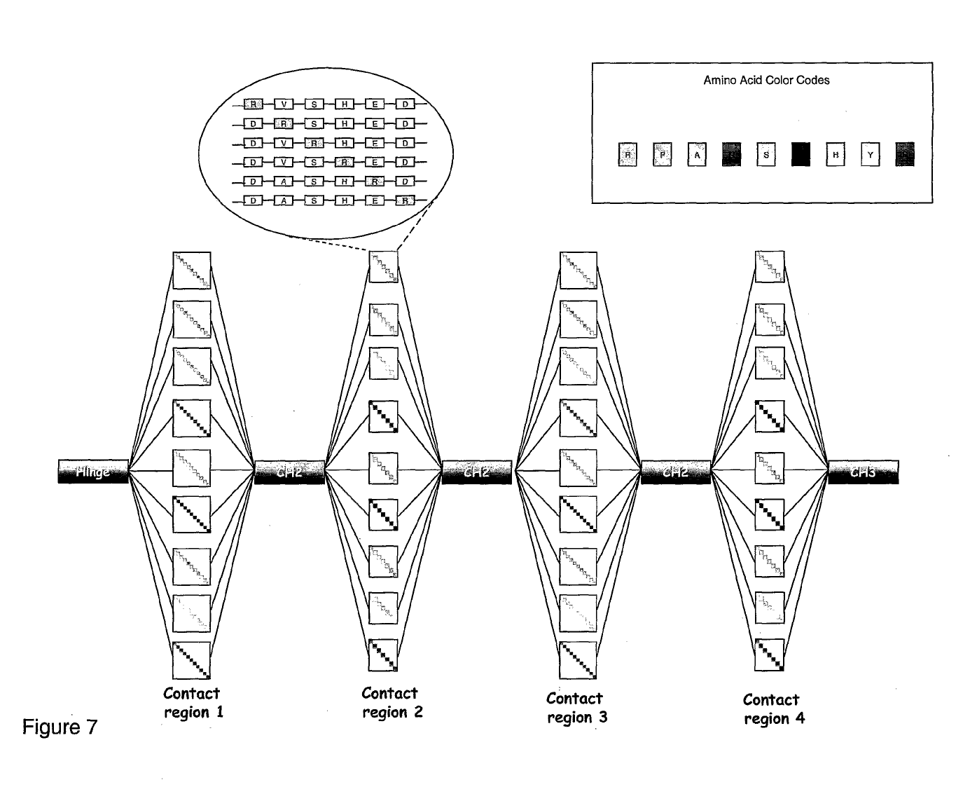

FIG. 7 is a representation of the various combinations available in combining the four Fc “contact” sub-regions where each “contact” sub-region is its' own nine LTM library. For example in one library, it can be composed of an asparagine LTM at one position in “contact” sub-region 1, aspartate LTM at one position in “contact” sub-region 2, tryptophan at one position in “contact” sub-region 3, and proline one position in “contact” sub-region 4. The library size, for a set of nine different amino acids, is thus 36⁴.

### B. Combinatorial Beneficial Mutagenesis (CBM) Libraries

After LTM Fc variants are screened and selected using functional assays, the rescue of those clones then allows for identification of that DNA coding sequences, as will be detailed below. In the combinatorial beneficial mutation approach, coding sequences are subsequently generated which represent combinations of the beneficial LTM mutations identified and combines them together into a single library. These combinations may be combinations of different beneficial mutations within a single sub-region or between two or more sub-region within the Fc. Therefore, synergistic effects of multiple mutations can be explored in this process.

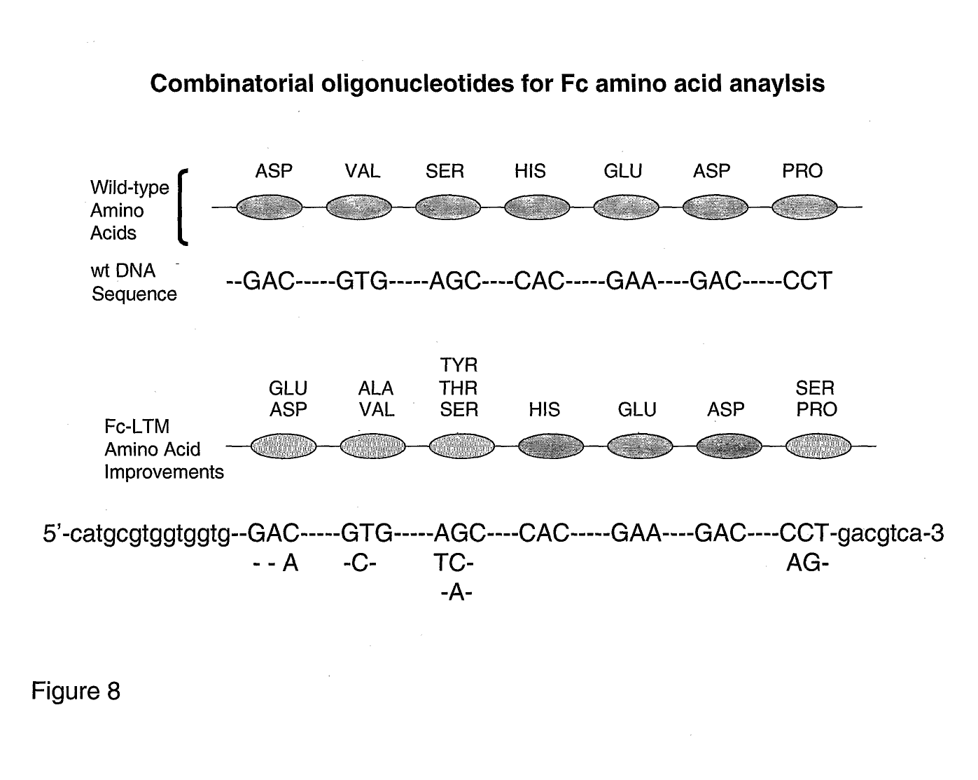

The combinatorial approach resembles the Walk Through Mutagenesis method (U.S. Pat. Nos. 5,798,208, 5,830,650, 6,649,340B1 and US20030194807) except that the selected codon substitutions within the Fc sub regions are the different beneficial amino-acid substitutions identified by LTM. As shown in FIG. 8, this coding-sequence library can be prepared by a modification of the WTM method, except that instead placing codons for a single amino acid at each different position in the variable coding region, the codons that are introduced are those corresponding to all beneficial mutations detected in the LTM method. Like WTM, not every residue position in the Fc CBM library will contain a mutation, and some positions will have multiple different amino acids substituted at that position. Overall, many if not all potential combinations of beneficial mutations will be represented by at least one of the coding sequences in the library.

### III. Generating Enhanced-Effector IgG₁ Fc Fragments

This section describes methods for generating and expressing Fc-LTM library Fc fragments in accordance with the invention. The design of oligonucleotide LTM and CBM libraries is preferably carried out using software coupled with automated custom-built DNA synthesizers. Implementation of the LTM and CBM strategies involves the following steps. After selection of target amino acids to be incorporated into the selected Fc region(s), the software determines the codon sequence needed to introduce the targeted amino acids at the selected positions. Optimal codon usage is selected for expression in the selected display and screening host, e.g., the mammalian expression system. The software also eliminates any duplication of the wild-type sequence that may be generated by this design process. It then analyzes for potential stop codons, hairpins, loops and other problematic sequences that are then fixed. The software determines the ratios of bases added to each step in the synthesis (for CBM) to fine tune the amino acid incorporation ratio. The completed LTM or CBM design plan is then sent to the DNA synthesizer, which performs automated synthesis of the primers of oligonucleotides used in generating a mutagenized gene.

### A. Construction of a Surface Expression Fc for LTM Analysis

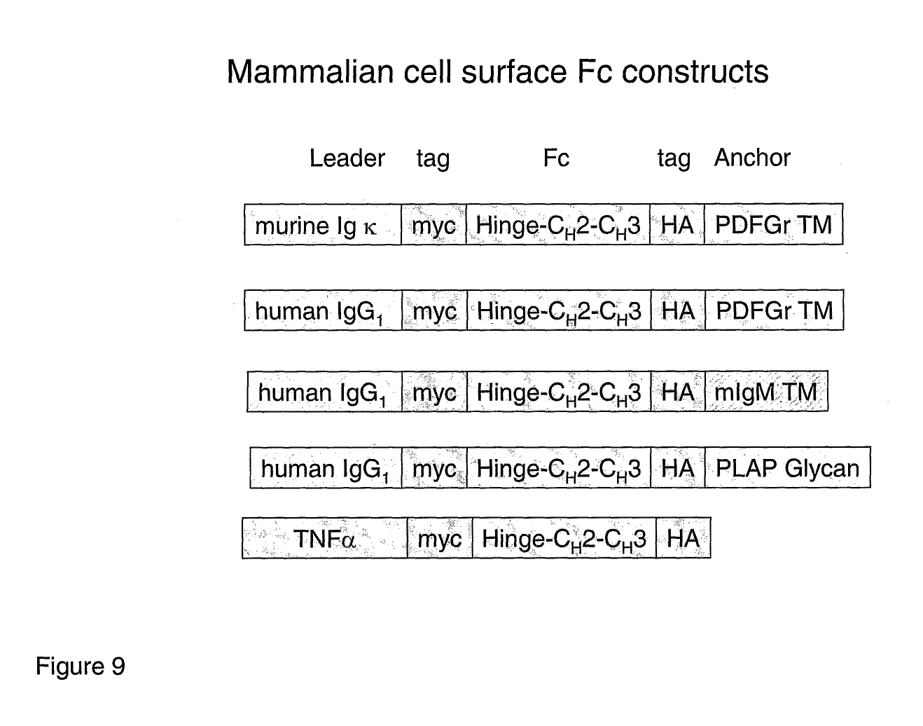

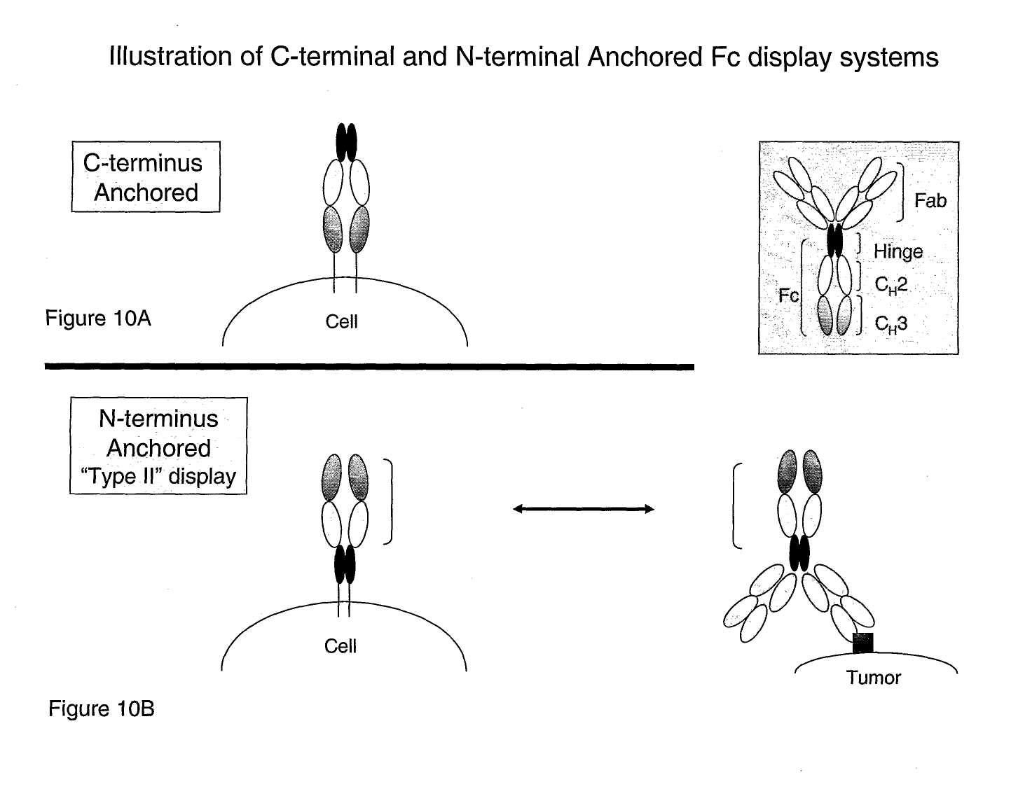

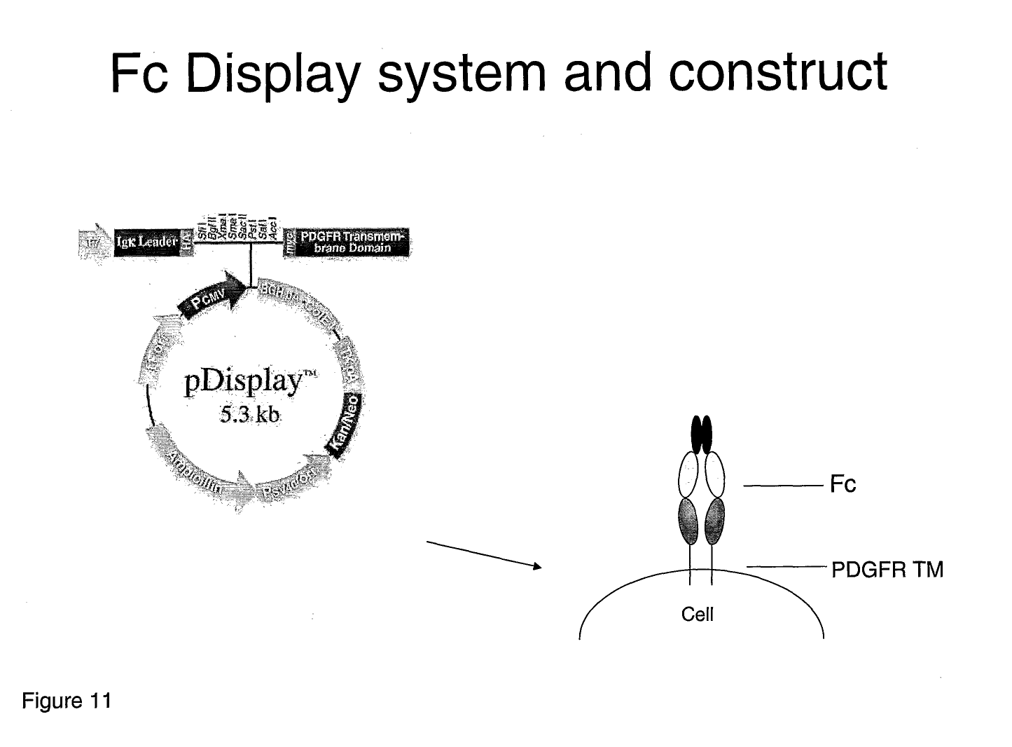

A wild type IgG₁ gene can be obtained from available sources and amplified by standard techniques (Example 1A). A chimeric surface expression Fc wild type gene construct (approximately 0.65 kb) can be assembled in vitro by SOE-PCR by fusing at the N-terminal, an extracellular export signal and at the C-terminus, a membrane anchoring signal. A list of potential N-terminal extracellular export signals include those from human IgG₁ and murine IgGₖ (SEQ ID:7). The list of potential C-terminal membrane anchoring signals include; placental alkaline phosphatase protein (PLAP), membrane IgM and Platelet Derived Growth Factor (PDGF) (SEQ ID: 8). The various fusion constructs are diagrammatically illustrated in FIG. 9. These components were PCR amplified and assembled as detailed in Example 1B. Various Fc surface expression constructs (FIG. 9) are possible in fusing an N-terminus murine IgGκ signal and C-terminus PDGF transmembrane (SEQ ID:9), an N-terminus human IgG₁ signal and C-terminus IgM transmembrane (SEQ ID:10), or an N-terminus human IgG₁ signal and C-terminus PLAP membrane lipid insertion signal (SEQ ID:11). In this iteration, the fusion construct has the C_{H}3 domain proximal (closest) to the cell membrane while the C_{H}2 domain is distal (FIG. 10A). FIG. 11 shows the pDisplay expression vector for cloning the Fc-LTM construct in between the N-terminal Igκ leader and C-terminal PDGF Receptor transmembrane anchor.

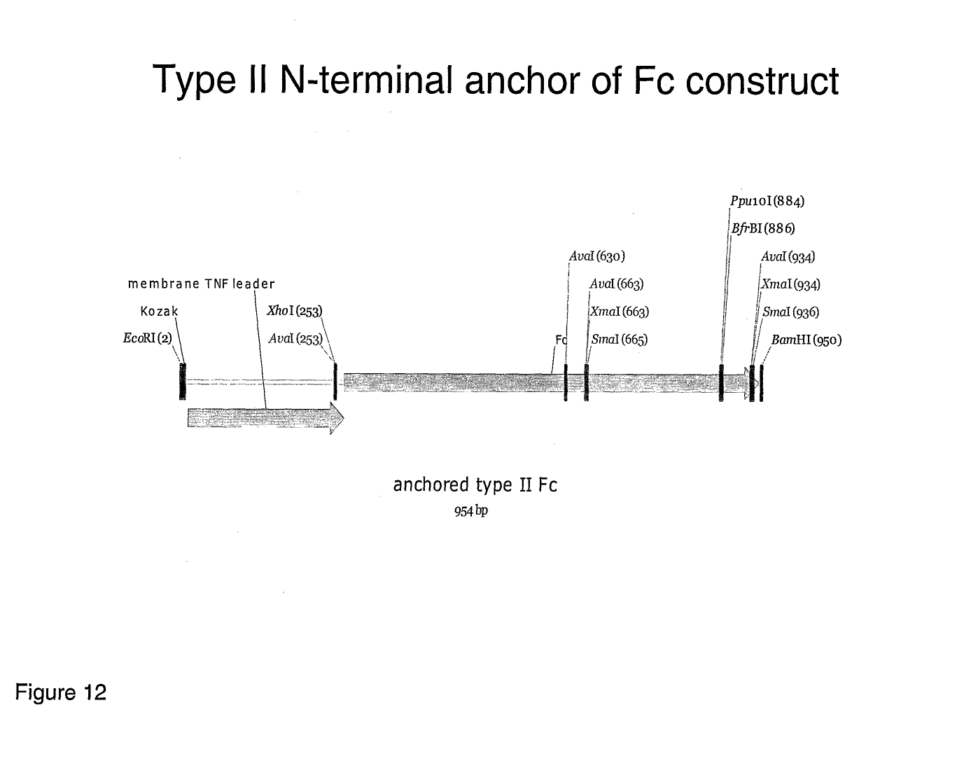

In some applications it may be desirable that the C_{H}2 domain is proximal to the cell surface membrane and the C_{H}3 is distal (FIG. 10B) as it mimics the natural presentation of IgG target binding. The following vector for this alternative orientation has been designed by fusing an N-terminal trans-membrane leader/anchoring signal sequence to precede the Fc gene region (FIG. 12), as detailed in Example 1C.

### B. Preparation of Fc-LTM Libraries by Kunkel Mutagenesis

The Fc-LTM libraries used in the invention are prepared by Kunkel mutagenesis of the Fc expression construct prepared in Section A above, and as detailed in Example 2. A single-stranded Fc template for Kunkel was prepared as in Example 2A. Kunkel mutagenesis of the template was carried out according to standard methods, as detailed, for example, in Kunkel, T. A. (1985) Proc. Natl. Acad. Sci. USA 82:488-92; Kunkel, T. A. et al. (1987) Meth. Enzymol. 154: 367-82; Zoller, M. J. and Smith, M. (1983) Meth. Enzymol. 100:468-500; Hanahan, D. (1983) J. Mol. Biol. 166:557-80; and Maniatis, T., Fritsch, E. F. and Sambrook, J. (1989) in Molecular Cloning, A Laboratory Manual.

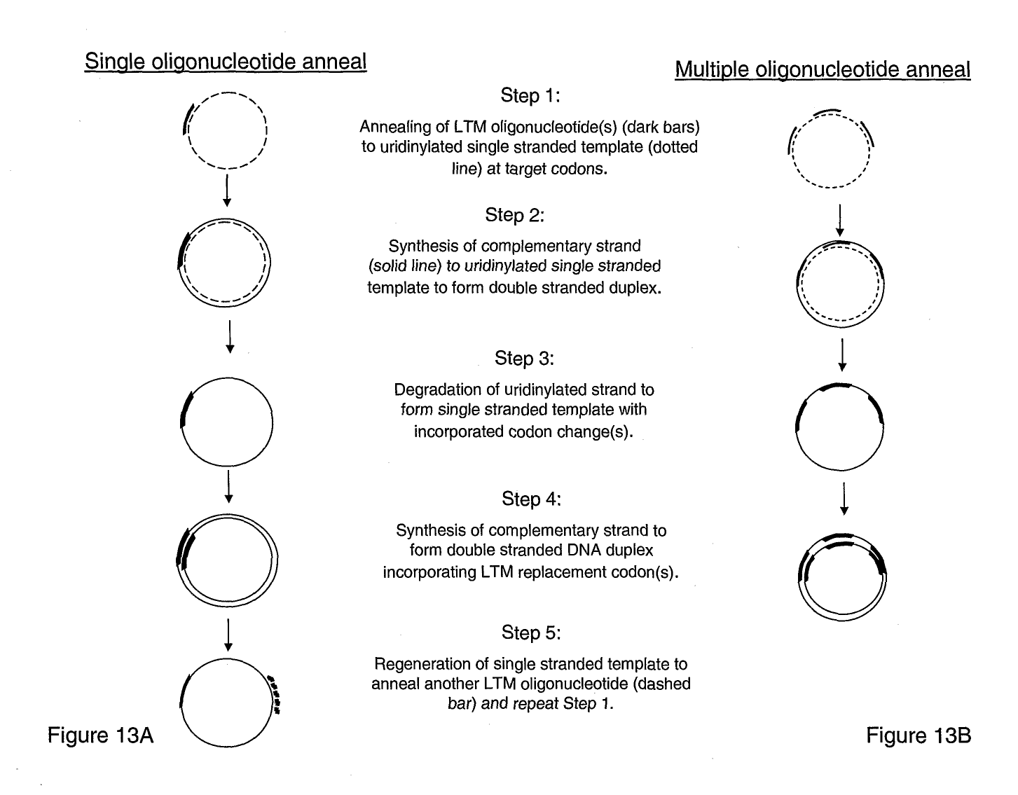

FIG. 13A shows general steps in the Kunkel mutagenesis for introducing a single codon substitution into a template wildtype Fc coding sequence. Initially, the single-stranded uridinylated template (dashed-line circle in Step 1) is reacted with an oligonculeotide (solid fragment) that carries a selected codon substitution for a selected position in the C_{H}2 and/or C_{H}3 domain of the gene under hybridization conditions (Step 1 in FIG. 13A). After synthesis of the complementary strand (solid line in Step 2) to form a double-stranded duplex, the uridinylated strand is degraded to yield a single stranded template with the incorporated codon substitution change (Step 3). This stranded is used to synthesize the double-stranded form of the mutated gene (Step 4). To introduce additional mutations into the mutated gene, the double stranded gene is manipulatd to regenerate a uridinylated single stranded template (Step 5), with addition of another oligonucleotide at a new position on the gene. For example, the two regions may represent the C_{H}2 and C_{H}3 domains of the Fc coding sequence, or may represent two of the four contact regions of the C_{H}2 domain.

In practice, a single reaction scheme such as illustrated in FIG. 13A is carried out by adding to a template, different oligonucleotide whose codon substitutions represent all of the individual amino acid substitutions at each position within a given region of the gene. For example, to introduce LTM mutations for each of nine amino acids at each of five positions in an Fc region, a total of 45 different oligonucleotides would be added to a single reaction mixture. After conducting steps 1-5, a sufficient number of the reaction products are checked to confirm the presence of the different LTM sequences desired. For example, to confirm the presence of all 45 different sequences in the above example, to may be sufficient to sequence 20-30 sequences to demonstrate that the different sequences are each represented in the mixture.

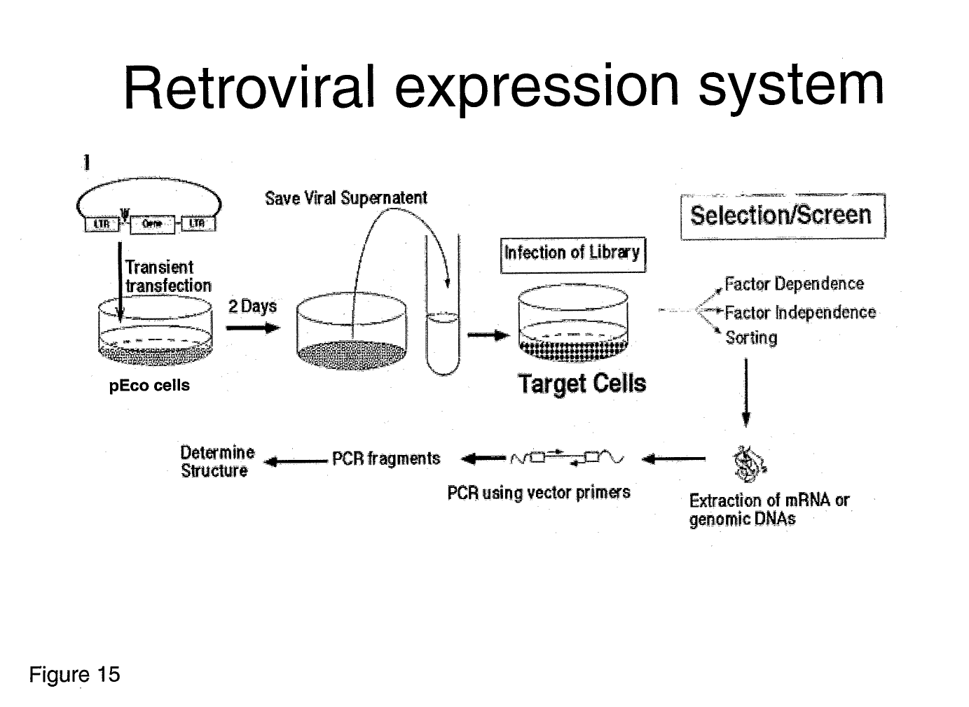

Double, triple and quadruple regional LTM libraries can be created as above but instead of using the wild type Fc gene as the Kunkel template, a previously generated LTM library template is chosen instead. To create a double LTM library for both “contact” sub regions 1 and 3, previously generated LTM “contact” sub region 1 mutant genes are used as single stranded templates to which are annealed a set of sub region 3 oligonucleotides to generate the double LTM library. The double LTM library can then be used as templates to incorporate LTM “contact” sub region 4 oligonucleotides to make the triple LTM libraries. By progressively utilizing the starting single and double LTM libraries, more complex arrays of LTM library can be developed using all the iterations of the LTM amino acids (FIG. 15A).

FIG. 13B illustrates a novel application of the Kunkel method, in accordance with one aspect of the present invention, for generating multiple mutations in each of a library of Fc coding regions. In this approach, separate sets of oligonucleotides (in the figures, three sets), each corresponding to a selected region of the Fc gene, are added to the Fc template in Step 1. For example the three sets of oligonucleotides used in the method could correspond to the 36, 45, and 27 different sequences employed for L™ at the first three cojntact positions in the C_{H}2 domain. As seen, the first step of the method results in single-strand uridinylated template strands having one member from each set of codon-substitution mutations bound. By carrying out the same Steps 2-4 described above, the method results in the generation of double stranded Fc coding regions, each containing some combination of single selected mutations at each of the three Fc coding regions targeted. Details of the sequences in the actual Fc-LTM libraries are given in Example 2C.

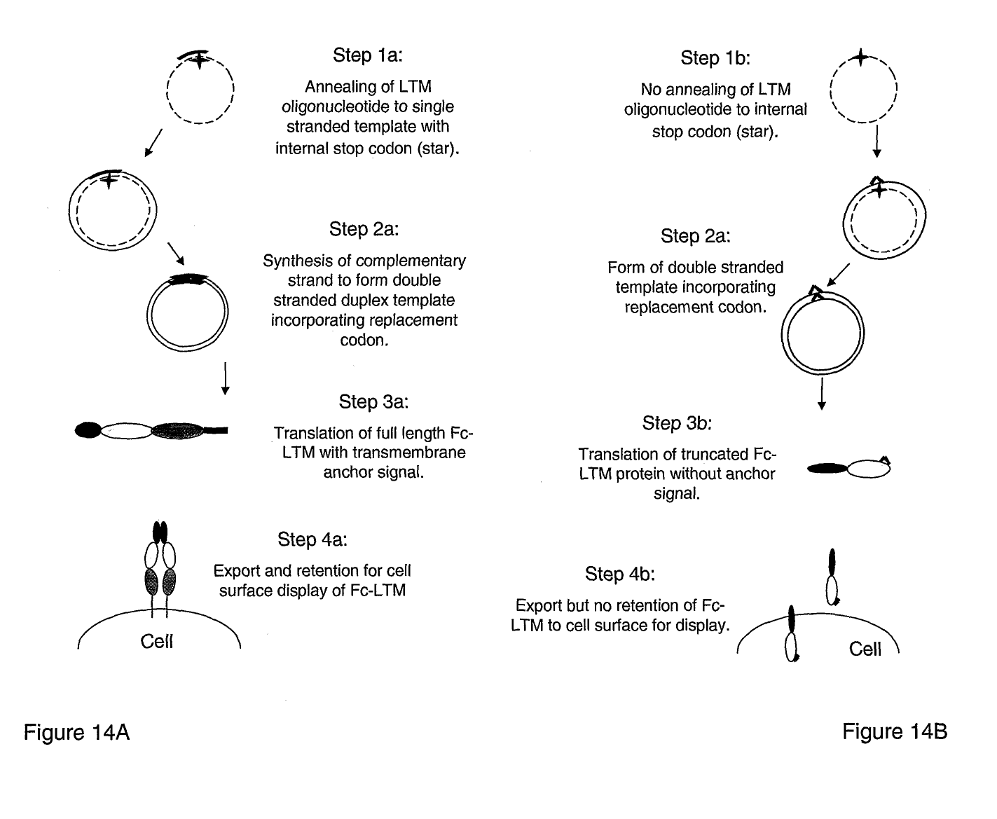

Prior to the Kunkel LTM mutagenesis, the Fc domain may be modified to introduced a stop codon into the reading frame in the various sub-regions to be examined by LTM. For example in regional Fc-FcγRIIIa “contact” point LTM library, there are four separate “stop-modified” templates. The wild type Fc template was “stop-modified” using the oligonucleotides shown in SEQ ID: 28. The purpose is that a “stop-modified” wild type template, which did not undergo Kunkel mutagenesis, will be expressed as an N-terminal truncated protein. These truncation constructs will be composed of an extracellular signal leader and varying lengths of the Fc domain. However, translation of the non-mutagenized reading frame will not continue through to the trans-membrane anchoring signal. Therefore, the “stop-modified” templates will be translated, exported but will not be retained on the extracellular cell surface (comparing FIGS. 14A and 14B). As such, library cells with truncations will not be recognized with subsequently added Fc receptors and binding proteins.

These “stop modified” templates allow a supplementary feature of re-introducing the wild type coding sequence. The addition of “open reading frame” oligonucleotides (SEQ ID: 29) allows the stop codon to be replaced with the original Fc codon. In this manner, the “wild type” re-introduction mutagenesis is proportional to that being introduced by the LTM oligonucleotides. The Fc-LTM surface expression libraries will therefore have an internal wild-type reference control that is not in relative overabundance.

Once the Fc template is LTM modified, the construct is excised from the cloning vector, purified, and ligated into a suitable expression vector (e.g., Clontech, Palo Alto, Calif.). Following E. coli transformation and selection on LBamp plates, the constructs may be sequenced to confirm the Fc desired coding changes and the adjacent extracellular secretion and membrane targeting regions.

### C. Expression of Fc LTM Libraries

A variety of methods for selectable antibody expression and display are available. These include biological “particles (cells or viral particles) such as bacteriophage, Escherichia coli, yeast, and mammalian cell lines. Other methods of antibody expression may include cell free systems such as ribosome display and array technologies which allow for the linking of the polynucleotide (i.e., a genotype) to a polypeptide (i.e., a phenotype) e.g., Profusion™ (see, e.g., U.S. Pat. Nos. 6,348,315; 6,261,804; 6,258,558; and 6,214,553).

One preferred expression system includes' a mammalian cell that is (i) capable of producing clinical-grade monoclonal antibodies, (ii) nonadherent in culture, and (iii) readily transduced with retrovirus. Exemplary cells having these characteristics are BaF3, FDCP1, CHO, and NS0 cells.

These cells can be transduced with Fc library expression vectors according to known procedures. In the method detailed in Example 3, an pLXSN mammalian expression vector containing a promoter element, which mediates the initiation of transcription of mRNA, the Fc coding sequence, and signals required for the termination of transcription and polyadenylation of the transcript is transfected into the amphotropic packaging cell line PA317. FIG. 15 shows a transient transfection protocol where the viral supernatant is directly collected, as detailed in Example 3A and 3B. The expression cell line, e.g., NS0 cells, are transduced with the harvested viral supernatant as detailed in Example 3C. Expression of Fc fragments on the cell surfaces, and binding of Fc receptors, such as FcγRIIIA to the expressed polypeptide can be confirmed by FACS analysis, as described in Example 3D.

### IV. Screening Fc Fragments for Enhanced Effector Functions

This section considers methods for screening the expressed Fc fragments of the above Fc-LTM libraries for enhanced effector function. Subsection A below describes several Fc receptor proteins and indicates for each, desired changes (increases or decreases) in binding affinity that may be screened for. As noted in Section II, this effector function will be related to (i) a shift in binding affinity constant (K_{D}), with respect to a selected IgG₁ Fc binding protein, relative to native IgG₁ Fc; and/or (ii) a shift in the binding off-rate constant (K_{off}); with respect to a selected IgG₁ Fc binding protein, relative to native IgG₁ Fc. Thus, the expressed Fc libraries can be screened for a change in binding constant, which can be an increase or decrease in binding constant, depending on the binding constant being measured, the Fc binding protein involved, and the desired effect of the change in binding constant, as described below in Subsection B. Alternatively, and according to a novel screening method in the invention, the LTM library Fc fragments can be screened directly for an enhanced effector function related to CDC or ADCC, by measuring the extent of cell lysis directly in Fc-expressing cells, as disclosed in Subsection C. Specific receptor targets are given in Subsection D.

A. Fc receptors

This section considers various Fc receptor proteins (targets), and the therapeutic implications of achieving enhanced or reduced Fc binding to the proteins for the four main subclasses of IgG antibodies. Generally, if Fc mediated effector functions are to be enhanced, it is usually desirable to increase binding of IgG₁ and IgG₃ to those Fc receptors that mediate effector activity, such as the FcγRIIIa receptor. However, some applications require decreased binding to FcγR receptors of any type. For example, those IgGs of all isotypes having Fc fragments conjugated to cytotoxic payloads (radioactive-labels) would otherwise bring healthy FcγR bearing-immune cells in to the Fc-radio-conjugates and kill them. In other applications, it may be desirable to have a purely neutralizing antibody that has no effector function. In this circumstance, IgG₂ and IgG₄ have low affinity to most Fc receptors, but it may be desirable to further reduce Fc receptor binding to these isotypes. For example, IgG₄ binding to FcγRI could be further reduced, and IgG₂ binding to FcγRIIa could be reduced to minimize effector functions. IgG₃ has lower affinity for FcRN, and increasing affinity towards this receptor should increase the circulating half-lives of the antibody.

In the table below an up arrow ↑ is used to indicate an increased affinity of the Fc fragment for the associated Fc binding partner. This increased affinity can be achieved by an increased binding affinity constant K_{D}, or a decreased K_{off} rate constant. An increased binding affinity constant will reflect a change toward a smaller-valued number, e.g., 10⁻⁷ M to 10⁻⁸ M. A decreased K_{off} value will mean a lower-valued K_{off}, indicating that Fc-binding receptor complex has a reduced tendency to dissociate. Similarly, a down arrow ↓ in the table ↑ is used to indicate a decreased affinity of the Fc fragment for the associated Fc binding partner. This decreased affinity can be achieved by a decreased binding affinity constant K_{D}, or an increased K_{off} rate constant. A decreased binding affinity constant will reflect a change toward a larger-valued number, e.g., 10⁻⁸ M to 10⁻⁷ M. An increased K_{off} value will mean a higher-valued K_{off}, indicating that Fc-binding receptor complex has a greater tendency to dissociate. A sideways arrow → in the table means no (or substantially no) change in the binding affinity.

Considering the various Fc receptors listed in the table, C1Q is the complement binding complex present in plasma that plays an essential part in CDC, as described above. A target cell recognized by IgG antibody that binds C1q will direct complement mediated cell death (CDC). Increasing C1q affinity for IgG₁ and IgG₃ will increase CDC function (increasing K_{D} and/or decreasing K_{off}). Decreasing C1Q affinity for IgG₂ (decreasing K_{D} and/or increasing K_{off}) increasing can reduce unwanted effector activity involving IgG₂ antibodies receptor

IgG Fc Effector Table

| | IgG₁ | IgG₂ | IgG₃ | IgG₄ |
|---|---|---|---|---|
| C1q binding | ↑ | ↓ | ↑ | |
| FcγRI | ↑ | | ↑ | ↓ |
| FcγRIIa | ↑ | ↓ | ↑ | |
| FcγRIIb | ↓ | ↑ | ↓ | ↑ |
| FcγRIIIa | ↑ | | ↑ | |
| FcγRIIIb | ↓ | | ↓ | |
| FcRN | [symbol] /↑ | [symbol] /↑ | ↑ | [symbol] /↑ |
| Protein A | [symbol] ↑ | [symbol] /↑ | [symbol] ↑ | [symbol] /↑ |

The FcγRI receptor is a high affinity receptor found on monocytes, macrophages, neutrophils and functions in phagocytosis and ADCC. FcγRI has high affinity for IgG₁ and IgG₃, and increasing the affinity of IgG₁ and IgG₃ Fc's for FcγRI will increase ADCC function. The natural affinity of FcγRI for IgG2 and IgG4 is none or very low, respectively. Further decreasing the FcγRI affinity of IgG₂ and IgG₄ Fcs can reduce unwanted receptor interaction and unwanted effector activity.

FcγRII receptors (FcγRIIa, FcγRIIb, FcγRIIc) are found on B cells, platelets, basophils, eosinphils, neutrophils, monocytes and macrophages, and bind to IgG₁ and IgG₃ Fc fragments, but bind to IgG₂ and IgG₄ only weakly or not at all. FcγRIIa/c receptors are positive regulators of Fc functions; FcγRIIb receptor is a negative regulator involved in feedback inhibition of Ig production. Increasing the affinity of IgG₁ and IgG₃ Fc's for FcγRIIa/c will increase Fc mediated ADCC effector functions. Decreasing the affinity of IgG₁ and IgG₃ Fc's for FcγRIIb will lessen the feedback inhibition. Further, decreasing the affinity of IgG₂ Fc's for FcγRIIa/c will reduce ADCC stimulation of IgG2 isotype. Increasing the affinity of IgG₂ and IgG₄ Fcs for FcγRIIb will further negatively regulate ADCC activity.

The FcγRIII receptors (FcγRIIIa and FcγRIIIb) are high affinity receptors found on monocytes, macrophages, neutrophils and NK cells and functions in phagocytosis and Antibody Dependent Cellular Cytotoxicity (ADCC). FcγRIIIa is a positive regulator of Fc functions, and FcγRIIIb, a negative regulator as it performs no intracellular signaling. FcγRIII's have affinity for IgG₁ and IgG₃. Thus, increasing the affinity of IgG₁ and IgG₃ Fcs for FcγRIIIa will increase Fc mediated ADCC effector functions.

The FcRN receptor functions in the maintenance of constant IgG levels by removing IgG from circulation and recycling through the intracellular vesicles. FcRN has high affinity for IgG₁, IgG₂ and IgG₄ which, through recycling, allows for 3 week circulation ½ life. FcRN has a lower affinity for IgG₃ which results in a much shorter circulatory ½ life. Maintaining or increasing the FcRN affinity for IgG₁ and IgG3 will thus improve circulation half life of IgGs and promote extended IgG₁ and IgG₃ effector functions. In certain embodiments, it may be advantageous to have reduced half-lives. For example, it may be undesirable to have circulating radiolabeled antibodies, since it may cause non-specific toxicity to blood cells. Reduced binding to FcRN would allow faster clearance of the unbound radiolabeled antibody.

Protein A is an IgG-binding protein that allows affinity purification of antibodies from cell culture manufacturing. Maintaining or increasing the Protein A affinity for all IgG isotypes would permit better purification from other cellular and growth media components.

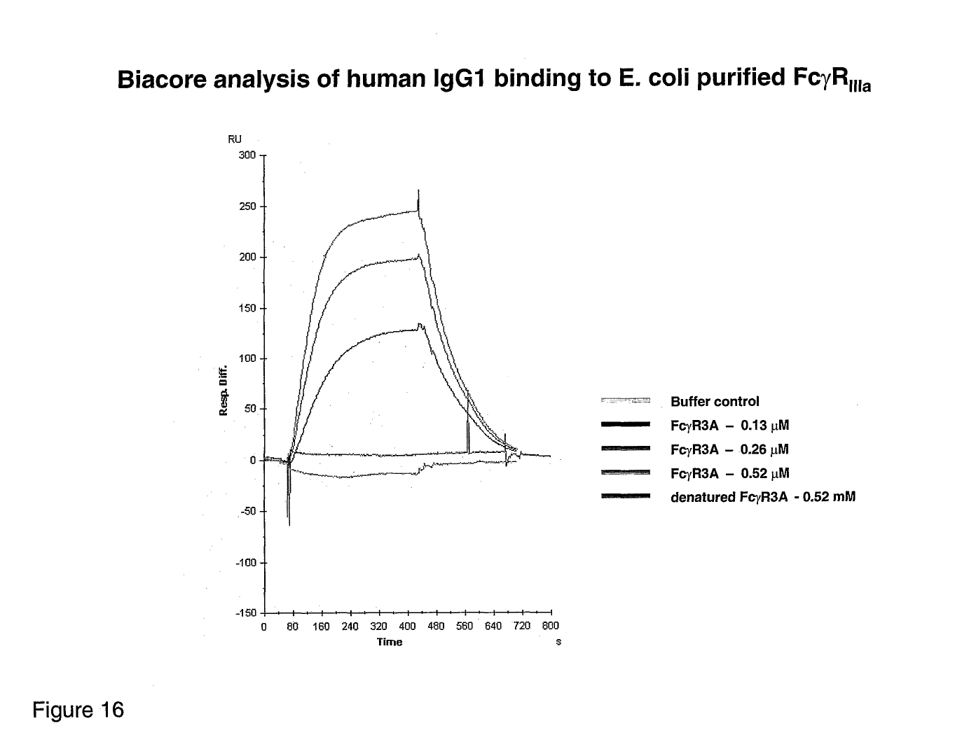

Example 4 described methods for obtaining or producing various Fc receptors in soluble form, for use in the screening assays described below for determining K_{D} or K_{off} values, and where appropriate, biotinylation of the receptor proteins. These include biotinylated Ciq (Example 4A), FcγRIIIa 176V and its polymorphic construct FcγRIIIa 176F, FcγRIIIa 176V, FcγRIIb and the polymorphs of FcγRIIa, FcγRIIIa176F and its polymorphic construct FcγRIIIa176V (Example 4B), and FcR receptor (Examples 4C-4E). BIAcore analysis was carried out to assess the functional IgG Fc binding and the preliminary affinities (K_{D}) of refolded FcγRIIIa fragments, as detailed in Example 5, with reference to FIG. 16. The BIAcore analysis is also consistent with known differences in binding affinity of IgG Fc with the V158 and F158 polymorphic forms of FcγRIIIa.

### B. Screening Fc-Producing Cells for Fc Fragments for Enhanced Binding Characteristics

This subsection will describe methods for screening Fc fragments produced by the Fc-LTM libraries for enhanced effector function, based on a desired change (increase or decrease) in either K_{D} or K_{off}. In either method, it is generally desirable to preselect cells for those expressing functional Fc fragments, that is, cells expressing Fc fragments cable of binding with at least moderate affinity to a selected Fc receptor.

### B1. Pre-selecting Cells to Enrich for Functional Fc

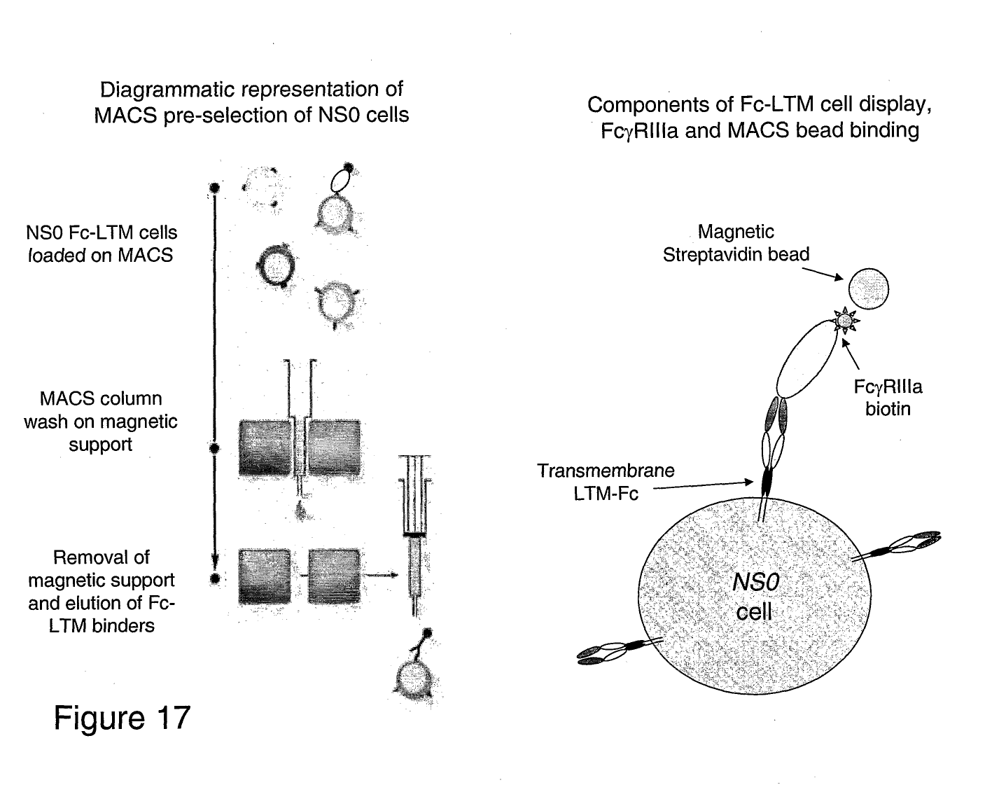

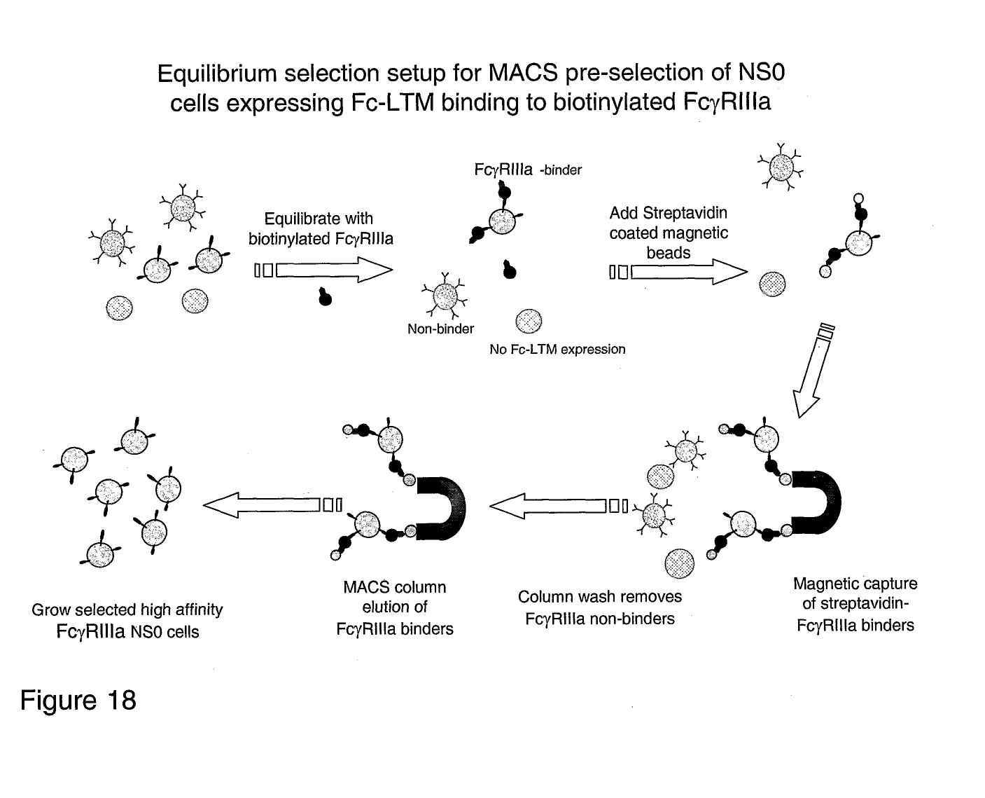

In the pre-selection method illustrated in FIGS. 17 and 18, Fc-expressing cells, e.g., NS0 cells, are incubated under equilibrium conditions with a biotin-labeled receptor, e.g., a biotin-labeled FcγRIIIa, and then streptavidin-labeled magnetic beads. As seen at the right in FIG. 17, cells expressing a function Fc receptor will form a “magnetic” cell-receptor-bead complex, whereas cells expressing non-functional Fc fragments will remain largely unreacted. The magnetically labeled cells are then separated from unreacted cells by placing a column containing the reaction mixture within a magnetic filed, as illustrated at the left in FIG. 17, and eluting unreacted cells. After removing the remaining cells mixture from the magnetic field, a cell population enriched for functional Fc fragments is eluted from the column.

The reaction steps involved in the pre-selection method are shown in FIG. 18. After equilibration of Fc-producing cells with biotin-labeled FcγRIIIa (upper middle frame), streptavidin-labeled particles are added (upper right), producing the cell-receptor complexes in cells producing functional Fc fragments. The magnetically labeled cells are separated from unlabeled cells by a column wash in a magnetic (MACS) column, followed by elution of the desired cells, and growing the enriched cells for subsequent selection based on Fc receptor binding affinity properties. Details of the pre-selection method are given in Example 6.

### B2. Screening Fc Fragments for Enhanced K_{D}

The pre-selection method illustrated in FIGS. 17 and 18 is also employed, with some modifications, for Fc fragments having increased (or decreased, depending on the receptor and desired therapeutic effect) binding affinity constants, i.e., increased (lower-valued) K_{D}. The method employs a selected biotinylated Fc receptor, e.g., a FcγRIIIa receptor and streptavidin coated magnetic beads to select high affinity molecules from mammalian-cell libraries.

Initially, the Fc-expression cells (typically pre-selected for functional Fc expression), are equilibrated with biotinylated FcγRIIIa, producing a mixture of cells having bound biotinylated FcγRIIIa, and low-affinity and non expressing cells. Following equilibration binding to FcγRIIIa, streptavidin coated beads are added to the mixture, forming a binding complex consisting of high-affinity expressing cells, biotinylated FcγRIIIa, and magnetic beads. The complexes are isolated from the mixture using a magnet, and the bound complex is washed several times under stringent conditions to remove complexes of low-affinity cells and non-specifically bound cells. The resulting purified complexes are released from the complexes, by treatment with a suitable dissociation medium, to yield cells enriched for expression of high-affinity Fc fragment.

In one exemplary screening method, the isolated cells are plated at low density, and clonal colonies are then suspended in medium at a known cell density. The cells are then titrated with biotinylated FcγRIIIa by addition of known amounts of FcγRIIIa, as indicated, e.g, from 10 pM to 1000 nM. After equilibration, the cells are pelleted by centrifugation and washed one or more times to remove unbound FcγRIIIa, then finally resuspended in a medium containing fluoresceinated spreptavidin. The fluoresceinated cells are scanned FACS to determine an average extent of bound fluorescein per cell. The Fc fragments selected will having a binding affinity that is preferably at least 1.5 higher, and typically between 1.5-2.5 higher (or lower, if decreased binding affinity is desired) than that of wildtype Fc fragments with respect to the selected receptor.

### B3. Screening Fc Fragments for Altered K_{off}

Alternatively, the Fc fragments expressed on the expression cells may be selected for enhanced K_{off}, i.e., a lower-valued K_{off}, where increased binding affinity is desired, or a higher-valued Koff, where reduced binding affinity is desired. The Fc fragments selected will preferably have K_{off} values that are at least 1.5 and up to 2-5 fold lower than the measured K_{off} for wildtype Fc fragment, when measured under identical kinetic binding conditions (or 1.5 to 2.5 fold higher if lower affinity Fc fragments are sought).

In the method for determining K_{off} values, Fc-expressing cells are incubated with a saturating amount of biotinylated Fc receptor, e.g., biotin-labeled FcγRIIIa, under conditions, e.g., 30 minutes at 25° C., with shaking, to effectively saturate displayed Fc fragment with bound receptor. The cells are then incubated with non-biotinylated FcγRIIIa at saturating conditions, for a selected time sufficient to reduce the percentage of biotinylated FcγRIIIa bound to the cells as a function of the off rate of the antigen. Following incubation, the cells are centrifuged, and washed to remove unbound biotinylated FcγRIIIa, yielding cells which contains a ratio of biotinylated and native FcγRIIIa in proportion of the antibody's K_{off}.

Details of the method are given in Example 7.

The k_{off} values are then determined by incubating the cells with a fluoresceinated streptavidin (streptavidin-PE) and a fluoresceinted cell marker (anti-his-fluorescein), washing the cells, and sorting with FACS. The k_{off} value is determined from the ratio of the two fluorescent markers, according to known methods. Example 7 provides additional details for the method.

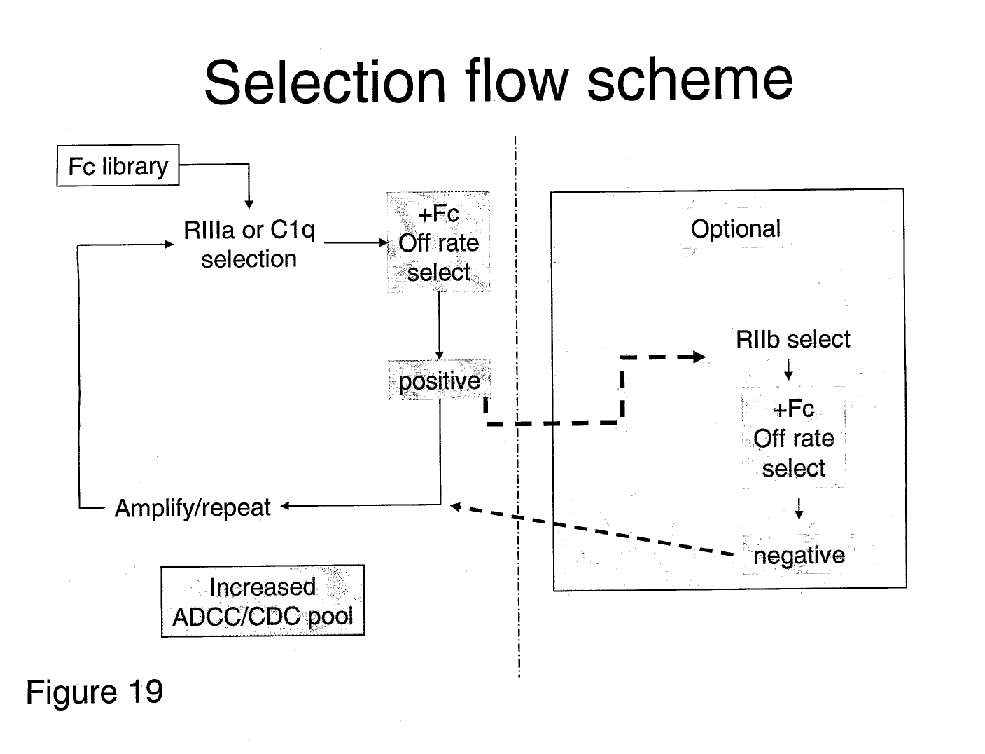

In some cases, it may be advantageous to select Fc fragments having-enhanced binding affinity for one Fc receptor and altered, e.g., decreased binding activity for a second Fc receptor. FIG. 19 shows a selection scheme for this type of selection. The left portion of the figure shows steps (which may be repeated one or more times) for selecting an Fc fragments having an enhanced K_{off} rate constant for an RIIIa receptor or C1Q complex, i.e., an Fc fragment having a lower-valued K_{off} value with respect to one of these Fc receptors. The Fc fragments from these clones will show increased CDC or ADCC activity when subsequently tested for cell-lytic activity in the CDC or ADCC assay. When a group of desired Fc-expressing clones are identified, these clones may be further for reduced binding affinity to a second Fc receptor, e.g., RIIb, employing similar methods, e.g., for screening cells for Fc fragments having higher-valued K_{off} constants with respect to target Fc receptor.

### B4. Cell Expansions and Determination of Enhanced Effector Sequences

After performing the binding affinity assay, those cells exhibiting a desired enhancement in Fc characteristics can be expanded for growth expansion. The Fc-LTM sequence from these clones are then “rescued” by PCR with Fc-LTM vector specific primers and subcloned into a suitable sequencing vector for sequence analysis and identification of the LTM amino acid change. Enhanced activity clones (either increased or reduced binding affinity with respect to a particular Fc receptor) thus identified may be further tested for actual effector function, e.g., in a CDC or ADCC assay of the type described below.

Exemplary receptors targets, and desired enhancement in binding affinity include one of: (i) an elevated binding affinity constant or reduced binding off-rate constant, with respect to Fc-binding protein C1q, FcγRI, FcγRIIa, and FcγRIIIa, (ii) a reduced binding affinity constant or elevated binding off-rate constant with respect to Fc-binding proteins FcγRIIb, FcγRIIIb; and an elevated or reduced binding affinity constant or a reduced or elevated binding off-rate constant, respectively, with respect to Fc-binding protein FcRN and protein A.

For some experiments, the method was used to monitor the quantitative ADCC effector differences in between individuals with either FcγRIIIa F158/V158 and/or FcγRIIa H131/R131 polymorphisms, as detailed in Experiment 9.

### B5. Combinatorial Beneficial Mutations

After the LTM Fc variants are screened and selected using functional assays, the rescue of those clones then allows for identification of that DNA coding sequences. In the combinatorial beneficial mutation (CBM) approach, coding sequences are subsequently generated which represent combinations of the beneficial LTM mutations identified and combines them together into a single library. These combinations may be combinations of different beneficial mutations within a single sub-region or between two or more sub-region within the Fc. Therefore, synergistic effects of multiple mutations can be explored in this process.

The combinatorial approach resembles the Walk Through Mutagenesis method (U.S. Pat. Nos. 5,798,208, 5,830,650, 6,649,340B1 and US20030194807) except that the selected codon substitutions within the Fc sub regions are the different beneficial amino-acid substitutions identified by LTM. As shown in FIG. 8, this coding-sequence library can be prepared by a modification of the WTM method, except that instead placing codons for a single amino acid at each different position in the variable coding region, the codons that are introduced are those corresponding to all beneficial mutations detected in the LTM method. Like WTM, not every residue position in the Fc CBM library will contain a mutation, and some positions will have multiple different amino acids substituted at that position. Overall, many if not all potential combinations of beneficial mutations will be represented by at least one of the coding sequences in the library.

### C. Direct Functional Screening

In accordance with one aspect of the invention, desired enhancements in effector function related to enhanced or inhibited CDC or ADCC can be screened directly, using Fc-expressing cells as the target cells for the screen. The method will be described with reference to FIGS. 1B and 1C, and is detailed in Example 8. The method will be described for screening expressed Fc fragments that enhance the level of CDC or ADCC. However, it will be appreciated how the method can be modified to select for Fc fragments having reduced or “neutralized” CDC or ADCC function.

FIG. 1B illustrates the events involved in cell-mediated cytotoxicity (CDC), which include initial binding of an antigen-specific antibody 26 to a cell-surface antigen 28 expressed on the surface of the cell, such as a tumor-specific antigen expressed on the surface of a tumor cell. With the antibody bound to the cell, binding of a C1q complement factor 32 to the antibody's Fc fragment 34 leads to cell lysis and destruction. This cell-lysis mechanism is aimed at removing potentially harmful cells from the body.

In the direct screening procedure, detailed in Example 8A and 8B, a pre-selected library obtained as above is diluted, and individual clonal cells placed in the wells of a microtitre plate, with a second “replica” plate being formed with the same cells. Human serum complement, including the C1q complex, is prepared as in Example 8B and added in serial dilutions to the microtitre plate wells, and the resulting CDC activity is measured fluorometrically. Those cells showing highest CDC levels, expressed in terms of amount complement added, may be identified as having a desired enhanced CD effector function., and/or may be expanded and re-screened for CDC activity until cells exhibiting a desired enhancement in CDC activity are identified. As above, when enhanced Fc fragments are identified, the associated cell-expression vectors can be analyzed to determine the Fc-coding sequence of the fragment.

The mechanism of cell lysis in of antibody-dependent cellular cytotoxicity ADCC),is illustrated in FIG. 1C. As in CDC, the mechanism involves the initial binding of an antigen-specific antibody 36 binding to a cell-surface antigen 38, such as a tumor specific antigen expressed on a tumor cell 40. The antibody's Fc fragment 42 can then bind to an Fc receptor protein 44, in this case an FcγRIIIa receptor, carried on a natural killer (NK) cell 46, leading to cell-mediated lysis of the tumor cell.

In the direct screening procedure, detailed in Example 8C, a pre-selected library obtained as above is diluted and individual clonal cells placed in the wells of a microtitre plate, with a second “replica” plate being formed with the same cells. To the microtitre plate wells are is added PBMCs including NK cells having surface-expressed receptor. After incubation, the cells are centrifuged and the cell supernatant assayed for released LDH, as detailed in Example 8C. Those cells showing highest levels of ADCC activity may be selected for enhanced Fc activity, and/or may be expanded and rescreened for ADCC activity until cells showing a desired enhancement in ADCC are identified.

After performing the Fc effector cell assays, those corresponding replica daughter wells exhibiting the desired level of ADCC or CDC activity can be expanded for growth expansion. The Fc-LTM sequence from these clones are then “rescued” by PCR with Fc-LTM vector specific primers and subcloned into a suitable sequencing vector for sequence analysis and identification of the LTM amino acid change.

After identification and sequencing of enhanced affinity Fc fragments, the identified sequences can be used, for example, in the construction of full length antibodies or single-chain antibodies having a selected antigen-binding specificity and an enhanced receptor function, e.g., an ability to enhance or suppress CDC or ADCC when administered to a subject, as discussed above. Example 10 described the construction of a full-length Rituxin antibody having enhanced CDC or ADCC function.

The following examples illustrate, without limitation, various methods and applications of the invention.

### Example 1

### A. Cloning of Wild Type IgG₁ Fc Gene

The wild type IgG₁ was obtained from (image clone #4765763, ATCC Manassas, Va.). The amino acid and DNA sequences of the individual C H2 and CH3 domains are shown in SEQ IDs:1-4 respectively. The IgG₁ Fc-gene (SEQ ID:5 and 6) was PCR amplified and cloned into pBSKII (Stratagene, La Jolla, Calif.) for propagation, miniprep DNA purification and production of single stranded DNA template (QIAgen, Valencia Calif.).

Fc domain PCR reactions were performed using a programmable thermocycler (MJ Research, Waltham, Mass.) and comprised of; Forward Fc PCR primer 5′-TAT GAT GTT CCA GAT TAT GCT ACT CAC ACA TGC CCA CCG T-3′, Reverse Fc PCR primer 5′-GCA CGG TGG GCA TGT GTG AGT,AGC ATA ATC TGG AAC ATC A-3′, 5 μl, of 10 uM oligonucleotide mix, 0.5 μl Pfx DNA polymerase (2.5 U/μl), 5 μl, Pfx buffer (Invitrogen, Calsbad, Calif.), 1 μl 10 mM dNTP, 1 μl 50 mM MgSO4 and 37.5 μl dH20 at 94° C. for 2 min, followed by 24 cycles of 30 sec at 94° C., 30 sec at 50° C., and 1 min at 68C and then incubated for a 68° C. for 5 min.

### B. Construction of Surface Expression Fc Gene for LTM Analysis

The chimeric surface expression Fc wild type gene construct (approximately 0.65 kb) was assembled in vitro by SOE-PCR by fusing at the N-terminal, an extracellular export signal and at the C-terminus, a membrane anchoring signal. A list of potential N-terminal extracellular export signals include those from human IgG₁ and murine IgGₖ (SEQ ID:7). The list of potential C-terminal membrane anchoring signals include; placental alkaline phosphatase protein (PLAP), membrane IgM and Platelet Derived Growth Factor (PDGF) (SEQ ID: 8). The various fusion constructs are diagrammatically illustrated in FIG. 9. Briefly, the IgGκ extracellular leader and HA-Tag sequences were PCR amplified using sense 5′-AGT AAC GGC CGC CAG TGT GCT-3′ and anti-sense 5′-GCA CGG TGG GCA TGT GTG AGT AGC ATA ATC TGG AAC ATC-3′ oligonucleotides from the pDISPLAY vector (FIG. 4, Invitrogen). The myc-tag and PDGF C-terminal membrane anchoring signals from pDISPLAY were amplified using sense 5′-TCC CTG TCC CCG GGT AAA GAA CAA AAA CTC ATC TCA GAA-3′ and antisense 5′-AGA AGG CAC AGT CGA GGC TGA-3′. The products of all three PCR reactions shared approximately 20 base pairs of overlapping complementary regions introduced by the neighboring upstream and downstream oligonucleotides.

The PCR products; N-terminal leader signal, Fc gene, and C-terminal membrane anchor section were then all incubated together as a mixture (5 μl of 10 uM oligonucleotide mix) and assembled by SOE-PCR using 0.5 μl Pfx DNA polymerase (2.5 U/μl), 5 μl Pfx buffer (Invitrogen), 1 μl 10 mM dNTP, 1 μl 50 mM MgSO4 and 37.5 μl dH20 at 94° C. for 2 min, followed by 24 cycles of 30 sec at 94° C., 30 sec at 50° C., and 1 min at 68° C. and then incubated at 68° C. for 5 min. The SOE-PCR assembly reaction permitted oligonucleotide overlap annealing, base-pair gap filling, and ligation of separate DNA fragments to form a continuous gene. The Fc DNA from the PCR reaction was then extracted and purified (Qiagen PCR purification Kit) for subsequent Xho I and EcoRI restriction endonuclease digestion as per manufacturer's directions (New England Biolabs, Beverly Mass.). The chimeric Fc surface expression construct was then subcloned into pBSKII vector and sequenced to verify that there were no mutations, deletions or insertions introduced. Once verified, this chimeric N-terminal leader signal, Fc gene, and C-terminal membrane anchor surface expression construct served as the wild type template for the subsequent strategies of building Fc-LTM libraries.

Various Fc surface expression constructs (FIG. 9) are possible in fusing an N-terminus murine IgG₁ signal and C-terminus PDGF transmembrane (SEQ ID:9), an N-terminus human IgG₁ signal and C-terminus IgM transmembrane (SEQ ID:10), or an N-terminus human IgG₁ signal and C-terminus PLAP membrane lipid insertion signal (SEQ ID:11). In this iteration, the fusion construct has the CH3 domain proximal (closest) to the cell membrane while the CH2 domain is distal (FIG. 10A).

### C. Construction of Surface Expression Fc Gene Type II Display

In some applications it may be desirable that the CH2 domain is proximal to the cell surface membrane and the CH3 is distal (FIG. 10B) as it mimics the natural presentation of IgG target binding. We have designed the following vector for this alternative orientation by fusing an N-terminal trans-membrane leader/anchoring signal sequence to precede the Fc gene region (FIG. 12). Potential N-terminal signal anchors can include those from Type II transmembrane proteins such as TNF-α (SEQ ID:37 and 38). TNF-α normally possesses 76-residue leader sequence required for translocation across the endoplasmic reticulum membrane (ER) for extracellular display. However this TNF leader/anchoring signal also possesses a natural proteolytic cleavage site to release TNF from the cell. We first modified the TNF proteolytic signal by deletion so that any Fc fusion construct would not be cleaved and released after membrane export. The N-terminal TNF-Fc gene fusion was constructed as above using SOE-PCR and appropriate oligonucleotide primers as illustrated in SEQ ID: 38. The chimeric N-terminal TNF-Fc gene sequences were then verified by DNA sequencing.

### Example 2

### A. Preparation of Fc Single Stranded Template for Kunkel Mutagenesis

All the above Fc expression constructs were cloned in PBSKII for the preparation of Fc single stranded DNA. The E. coli hosts CJ236 were grown in 2YT/Amp liquid medium until the OD600 reached approximately 0.2 to 0.5 Absorbance Units. At this timepoint, 1 mL of M13 K07 helper phage was added to the bacterial culture for continued incubation at 37° C. After 30 minutes, the bacteria and phage culture was transferred to a larger volume of 2YT/Amp liquid medium (30 mL) containing 0.25 ug/mL Uridine for overnight growth.

The next day, the culture medium was clarified by centrifugation (10 min at 10000 g) after which the supernatant was collected and ⅕ volume of PEG-NaCl added for 30 minutes. The mixture was further centrifuged twice more but after each centrifugation, the supernatant was discarded in favor of the retained PEG/phage pellet. The PEG/phage pellet was then resuspended in PBS (1 mL), re-centrifuged (5 min at 14 000 g). The supernatant was collected and then applied to DNA purification column (QIAprep Spin M13, Qiagen) to elute single stranded wild type IgG₁ Fc uridinylated-NA.

### B. Look Through Mutagenesis (LTM) Oligonucleotides

Synthetic oligonucleotides were synthesized on the 3900 Oligosynthesizer (Syngen Inc., San Carlos, Calif.) as per manufacturer directions and primer quality verified by PAGE electrophoresis prior to PCR or Kunkel mutagenesis use. LTM analysis introduces a predetermined amino acid into every position (unless the wildtype amino acid is the same as the LTM amino acid) within a defined region (US2004020306). In contrast to other stochastic mutagenesis techniques, the LTM oligonucleotide annealed to uridinylated single stranded template and is designed to mutate only one defined Fc amino acid position.

### C. Fc Domain Kunkel Mutagenesis with LTM Oligonucleotides

As described in the specification above, there are two Fc libraries constructed for L™ analysis. The first embodiment is being termed an “unbiased” C_{H}2×C_{H}3 library where each amino acid position in the Fc region will be replaced by the nine chosen LTM amino acids (FIG. 6). In total there are 1926 LTM oligonucleotides (214 Fc domain amino acids×9 LTM amino acid replacements per Fc position) and are on average, 63 base pairs in length. For the “unbiased” Fc domain library, the C_{H}2 (SEQ ID:1) and C_{H}3 (SEQ ID:2) regions were artificially divided into juxtaposed subsections of 5 to 7 amino acid length (SEQ IDs:12 and 13 respectively). The 18 CH2 and 16 CH3 subsections thus individually represent portions of the contiguous full length IgG₁ Fc sequence.

The second Fc LTM library represents the four separate IgG₁ Fc-FcγRIIIa “contact” points as identified from the IgG₁ Fc-FcγRIIIa co-crystal structure (FIG. 2A). This second library then delineates four sub-regions (SEQ ID:14-17) within the total “unbiased” CH2×CH3 library above. Therefore, the four “contact” sub-region LTM library is simply a subset of the “unbiased” C_{H}2×C_{H}3 LTM variants generated above. The desired amino acid replacements at “contact” sub-region 1 are shown in FIG. 2B. This “contact” sub-region 1: LLGG (SEQ ID:14) is coded for by the DNA sequence: CTG CTG GGG GGA and flanked by the DNA sequences 5′-cca ccg tgc cca gca cct gaa and ccg tca gtc ttc ctc ttc ccc cca aaa ccc-3′ framework. The four glycine LTM replacement oligonucleotides for “contact” sub-region 1 are listed (SEQ ID:18). The LTM oligonucleotide sequence: 5′-cca ccg tgc cca gca cct gaa GGG CTG GGG GGA ccg tca gtc ttc ctc ttc ccc cca aaa ccc-3′ demonstrates the glycine replacement codon (in bold). For “contact” sub-region 1, the remaining corresponding LTM oligonucleotides for asparagine (SEQ ID: 19), aspartate (SEQ ID: 20), histidine (SEQ ID: 21), tryptophan (SEQ ID: 22), iso-leucine (SEQ ID: 23), arginine (SEQ ID: 24), proline (SEQ ID: 25), and serine (SEQ ID: 26) show similar sequence design strategy. FIG. 3 illustrates the 4 LTM oligonucleotides for isoleucine. FIG. 17 is a representation of the various combinations available in combining the four Fc “contact” sub-regions where each “contact” sub-region is its' own nine LTM library. For example in one library, it can be composed of an asparagine LTM at “contact” sub-region 1, aspartate LTM at “contact” sub-region 2, tryptophan at “contact” sub-region 3, and proline “contact” sub-region 4.

In the example of the “unbiased” C_{H}2×C_{H}3 library, five glycine LTM replacement oligonucleotides (SEQ ID:27) are used to perform similar substitutions of at the first sub-region of the C_{H}2 domain defined by the amino acid sequence LLGGPSV (SEQ ID: 12). FIG. 18 is then an example of “unbiased” CH2 sub-region 8 with an aspartate LTM in conjunction with a “unbiased” CH3 sub-region 1 histidine LTM. Hereafter, the libraries constructed as above, whether “contact” sub-region or “unbiased” C_{H}2×C_{H}3 sub-region will be referred to as “Fc-LTM” libraries.

### Example 3

### A. Retroviral pLXSN Construction and Viral Particle Harvesting

The pLXSN mammalian expression vector contains one promoter element, which mediates the initiation of transcription of mRNA, the polypeptide coding sequence, and signals required for the termination of transcription and polyadenylation of the transcript. PLXSN contains elements derived from Moloney murine leukemia virus (MoMuLV) and Moloney murine sarcoma virus (MoMuSV), and is designed for retroviral gene delivery and expression.

Briefly, the pLXSN/Fc construct is transfected into the amphotropic packaging cell line PA317 (or other alternative cells) by calcium phosphate precipitation (Gibco, Carlsbad, Calif.). FIG. 14 shows a transient transfection protocol where the viral supernatant is directly collected. For stable cell lines, the transfectants are selected by culturing the cells for 2 weeks in complete DMEM containing G418 (Gibco) at a concentration of 800 μg/ml. The antibiotic selection can obtain a population of cells that stably expresses the integrated vector. If desired, separate pLXSN/Fc variant viral particle-producing PA317 clones can be isolated from this population and positively identified by reverse transcription (RT)-PCR (for both neomycin resistance gene and Fc mRNAs). Positive pLXSN/Fc clones are then expanded in DMEM and virus-containing supernatant is harvested to infect murine NS0 cell line (Sigma), CHO-K1 (ATCC, Manassas, Va.). When the retroviral supernatant is ready for harvesting, the supernatant is gently remove and either filter through a 45 μM filter or centrifuged (5 min at 500 g at 4° C.) to remove living cells. If the retroviral supernatant is to be used within several hours, it can be kept on ice. Otherwise, the retroviral supernatant may be frozen and stored at −70° C. Thawed retroviral supernatant is ready for immediate use in subsequent experiments.

### B. Transient Transfection and Harvesting of Viral Supernatant for NSO Transduction

The ecotropic cell line pECO (Clontech) is grown in Growth Medium (DME containing 10% heat inactivated fetal bovine serum, 100 U/ml Penicillin, 100 U/ml Streptomycin, 2 mM L-Glutamine). The following procedure is illustrated in FIG. 14. One day prior to transfection, the cells are seeded on plate and evenly distributed to subconfluency (50-60%). Subconfluent cells can be transfected using either conventional calcium phosphate protocols or cationic lipids such as Lipofectamine (Invitrogen). Briefly, to transfect cells in one plate, 125 μl Opti-MEM is mixed with 5 μl Lipofectamine 2000 and left to sit for 5 min (RT). In a separate reaction, 125 μl of Opti-MEM mixture is added to approximately 5 μg DNA. These two solutions are then combined and allowed to sit for 20 min before addition to the cells. The transfection reagent and cells in growth medium is then incubated overnight at 37° C. The following day, the overnight media is replaced with fresh GM. Two days (48 hours) post-transfection, the cell culture supernatant is collected into 15 ml tubes and centrifuged (5 min at 2000 g) to pellet debris.

For suspension cells such as NSO, a mouse myeloma cell line with lymphoblastic morphology, the cells are grown to log phase growth to approximately 5×10⁵ cells/ml. The NS0 cells are pelleted after a brief centrifugation and resuspended in 1 ml of fresh media containing diluted retroviral supernatant (>100 folds) and incubate for 12-24 hours at 37° C. A series of test dilutions can be performed with the retroviral supernatant to optimize transduction efficiency. NS0 library cells can then be monitored for transduction efficiency and Fc-LTM expression by subsequent FACS analysis.

### C. Infection of Non-Adherent Cells by Addition of Retroviral Supernatant

Murine tumor cell line NS0 is transduced with the harvested pLXSN/Fc retroviral vector supernatants (transient system shown in FIG. 14). Briefly, an infection cocktail is prepared consisting of: RPMI growth medium, retroviral supernatant (fresh or thawed) and Polybrene (2 μg/ml) such that the total volume is 3 mls. Exponentially growing NS0 target cells are centrifuged (5 min at 500 g) and resuspended in the infection cocktail at a concentration of 10⁵-10⁶ cells per ml. Twenty four hours post-infection, the NS0 cells are centrifuged and resuspended in RPMI growth media for normal growth for an additional 24-48 hours before assay. RPMI growth media is with 10% defined calf serum (Hyclone, Logan, Utah) in RPMI with 2 mM L-glutamine, 100 U/ml of penicillin (Sigma-Aldrich, St. Louis, Mo.), 100 ug/ml of streptomycin, 1 mM sodium pyruvate and 1× non-essential amino acids (all supplements from Bio-Whitaker).

### D. FACS Analysis of Fc-LTM Variant Surface Expression

The essential goal in our screening process is for each mammalian cell to express LTM Fc-fusion protein on its cell surface. Surface expression of Fc can be determined by anti-human anti-Fcγphycoerytherin antibody, or by also staining for the Myc or HA tags (all PharMingen, San Diego, Calif.) and confirmed by flow cytometry. pLXSN/Fc NS0 transduced cells are collected by low speed centrifugation (5 mins at 500 g), washed twice with CSB (PBS and 0.5% BSA), resuspended, and then incubated with soluble anti-Fcγ-PE antibody. After 1 hour (in the dark, covered and on ice) the cells are twice washed with cold CSB and resuspended at a concentration of 10×10⁶ cells/mL. Negative control cells are NS0 transduced with empty pLXSN vector and positive control cells are pLXSN with wild type Fc. The pLXSN-Fc transformed cells should show a significant shift in fluorescence, compared to empty pLXSN vector. The cells are then analyzed on FACSscan (Becton Dickinson) using CellQuest software as per manufacturer's directions.

After Fc surface expression on the LTM library cells is confirmed, the next task is to verify that the extracellular Fc constructs are capable of binding Fc receptors, namely FcγRIIIa and C1q. This is essential as the initial pre-selection procedures and subsequent Fc effector functional assays require Fc receptor association. To investigate, NS0 cells expressing the wild type Fc domain are collected as above and incubated with either labeled FcγRIIIa or C1q protein. The FcγRIIIa or C1q proteins can be either phycoreytherin or FITC fluorescently labeled or biotinylated as described below. For example, NS0 cells expressing Fc variants capable of binding biotin-C1q can then be counterstained with secondary streptavidin-PE and analyzed by FACS. Functional FC-LTM variants will bind the labeled FcγRIIIa and/or C1q protein and yield higher fluorescence readings. The protocols below describe the procedures to isolate, purify and biotin label FcγRIIIa or C1q proteins.

### Example 4

### Production and Purification of Fc Binding Proteins

### A. C1q Biotin Labeling

Bioactive C1q protein is composed as a heterotrimer [SEQ ID:30-32] and available commercially in a purified form (Calbiochem, San Diego, Calif.). Biotinylation of the C1q protein can be accomplished by a variety of methods however; over-biotinylation is not desirable as it may block the epitope-antibody interaction site. The protocol used was adapted from Molecular Probes FluoReporter Biotin-XX Labeling Kit (cat# F-2610). Briefly, C1q 1 μl of 0.9 mg/ml stock (Calbiochem), was added to 100 μl 1 M sodium bicarbonate Buffer at pH 8.3 and 9.4 μl of Biotin-XX solution (10 mg/ml Biotin-XX solution in DMSO). The mixture was incubated for 1 hour at 25° C. The solution was transferred to a micron centrifuge filter tube, centrifuged and washed repeatedly (four times) with PBS solution. The biotinylated-C1 q solution was collected, purified over a Sephadex G-25 column, and the protein concentration determined by OD 280.

B. E. coli Expression and Purification of Soluble FcγRIIIa, FcγRIIa, and FcγRIIb

The DNA sequence of FcγRIIIa176V was obtained from ATCC (SEQ ID: 33). The FcγRIIIa176F polymorphism construct was re-engineered by Kunkel mutagenesis as described above (SEQ ID: 34). The following E. coli purification protocol also pertains to the extracellular domain of FcγRIIb (SEQ ID: 35 and 36) and FcγRIIa (SEQ ID: 40, 41 and 42). FcγRIIIa176F and FcγRIIIa176V were cloned into pET 20b expression vector (Invitrogen, Carlsbad, Calif.) which appended a C-terminal 6×HIS tag to the protein. The pET 20b-FcγRIIIa V/F176 constructs were then transformed into BL21 E. coli host cells. Liquid cultures (LB-Amp) of E. coli cells were expanded from overnight small scale (5 mL) to 250 (mL) and upon reaching an absorbance value of (0.5@600 nm) the FcγRIIIa protein was induced with IPTG (0.5 mM) for 4 hours at 25° C. If not immediately used in the following purification scheme, growth cultures were subsequently pelleted and stored at −80° C. Cell pellets were then resuspended in 6 ml B-PER® II lysis Reagent (Pierce, Rockford, Ill.) by vigorous vortexing until they were without large visible aggregate clumpings. Once-uniformly suspended, the cells were gently shaken at RT for 10 minutes. After which, the cell lysis mixture was centrifuge (10 min at 10 000 RPM) to initially separate soluble proteins from the insoluble proteins. The extracellular domains of the FcγRIIa H/R131 polymorphisms were cloned in the same fashion.

### C. Denaturation of Inclusion Body Protein

The lysis supernatant was (collected and saved/discarded) while the pellet was again resuspended in 6 ml B-PER® II reagent. Lysozyme was added to the resuspended pellet at a final concentration of 200 μg/ml and incubated at RT for 5 minutes. The insoluble inclusion bodies were then collected by centrifugation (30 min at 10000 RPM). The resulting pellet was again resuspended in 15 ml of B-PER® II (approximately 1:20 pellet volume to B-PER dilution) and mixed by vigorous vortexing. The inclusion bodies were collected by centrifugation (15 min at 10 000 RPM). The steps of pellet resuspension, vortexing and centrifugation were repeated ten more times after which the final pellet of the purified inclusions bodies was saved and stored.

D. Ni-NTA Protein Purification under Denaturing Conditions

Purified inclusion body was thawed on ice and resuspended in 1.5 ml Buffer B [100 mM NaH₂PO₄, 10 mM Tris Cl, 8 M Urea, pH:8]. Taking care to avoid foaming, the suspension was slowly stirred for approximately 60 minutes (RT) or until lysis is completed (as observed when the solution becomes translucent). The mixture was centrifuged (15 min at 10 000 RPM) to pellet the cellular debris. The supernatant (cleared lysate) was then collected and added to it, 5 mL of Ni-NTA resin (Qiagen) and mixed gently (60 minutes at 4° C.). The lysate-resin mixture was carefully loaded into an empty column and wash with 100 ml Buffer B (pH:6.3). The recombinant protein was then eluted with 20 ml Buffer B (pH:4.5).

### E. Refolding of Ni-NTA Purified Protein

The Ni-NTA purified FcR protein, 3 mL from above, was added dropwise with stirring to refolding buffer [0.1 M Tris/HCl, 1.4 M arginine, 150 mM NaCl, 5 mM reduced glutathione, 0.5 mM oxidized glutathione, 0.1 mM phenylmethylsulfonyl fluoride, 0.02% NaN₃} over a 6 hour time period and then stirred for 72 hours. The renatured protein solution was then dialyzed against 4 L of dialysis buffer [0.1 M Tris/HCl, 5 M NaCl, 0.1 M MgCl₂.6H₂O] that was replaced with fresh buffer twice more before an overnight dialysis period. Ni-NTA resin (2 mL) was added to the renatured protein solution and then gently stirred for 60 minutes (RT). The lysate-resin mixture was carefully loaded into an empty column and wash with 100 ml wash buffer B (10 mM Tris/HCl, 300 mM NaCl, 50 mM imidazole, pH:8.0). The recombinant protein was then eluted with 10 ml elution buffer (10 mM Tris/HCl, 300 mM NaCl, 250 mM imidazole, pH:8.0).

### Example 5

### Biacore Analysis of Refolded FcγRIIIa Protein Binding to Human IgG₁-Fc

To assess functional IgG Fc binding and gauge the preliminary affinities (KD=k_{d}/kₐ=k_{off}/kₒₙ) of the refolded FcγR_{IIIa} fragments, BIAcore—2000 surface plasmon resonance system analysis was employed (BIAcore, Inc. Piscatawy, N.J.). The ligand, human full length IgG₁ (Calbiochem) was immobilized on the BIAcore biosensor chip surface by covalent coupling using N-ethyl-N′-(3-dimethylaminopropyl)-carbo-diimide hydrochloride (EDC) and N-hydrosuccinimide (NHS) according to manufacturer's instructions (BIAcore, Inc). A solution of ethanolamine was injected as a blocking agent.

For the flow analysis, FcγRIIIa was diluted in BIAcore running buffer (20 mM Hepes buffered Saline pH 7.0) into three concentrations of 0.13 □M, 0.26 □M, and 0.52 □M. The aliquots of FcγRIIIa were injected at a flow rate of 2 □l/minute for kinetic measurements. Dissociation was observed in running buffer without dissociating agents. The kinetic parameters of the binding reactions were then determined using BIAevaluation 2.1 software.

FIG. 13A displays BIAcore results from the FcγRIIIa binding to IgG₁. It is evident from these plots that the reconstituted FcγRIIIa binds the immobilized IgG as indicated by the RU increase (Kₒₙ) in comparison to the negative control of heat denatured protein. Furthermore, the RU increase was proportion to the FcγRIIIa protein concentration applied. The BIAcore profiles also displayed FcγRIIIa expected dissociation profiles.

We have also measured the k_{off} kinetic difference between FcγRIIIaV158 and the Fc□RIIIaF158 polymorphisms and are shown in the table below. These preliminary results are in agreement with other publications where the FcγRIIIaF158 polymorphism has lower affinity to IgG₁ Fc as demonstrated by a six-fold faster k_{off} kinetic.

| | Biacore measured |
|---|---|
| Fc receptor polymorphism | k_{off} (s⁻¹) |
| FcγRIIIa V158 | 0.0139 |
| FcγRIIIa F158 | 0.0858 |

### Example 6

### High Throughput Pre-Selection of Fc-LTM Variant Library by Magnetic Sorting

After growth culture, the NS0 Fc-LTM cells are labeled by incubating with biotinylated C1q at saturating concentrations (400 nM) for 3 hours at 37° C. under gentle rotation. To remove unbound biotinylated C1q, NS0 cells are then washed twice with RPMI growth medium before being resuspended 1.0×10⁵ cells/μl in PBS. A ratio of single cell suspension of approximately 10⁷ cells (100 μl) is mixed with 10 μl streptavidin coated or anti-biotin microbeads (MACS, Miltenyi Biotec) is incubated on ice for 20 minutes with periodic inversions. After low speed centrifugation, the mixture is then twice washed with buffer and resuspended in 0.5 mL. These procedures and cellular components are diagrammed in FIGS. 4A and 4B.

The cell suspension is applied to a LS MACS column placed in the magnetic field separator holder. The MACS column is then washed with 2×6 mL of buffer removing any unbound cells in the flow-through. The MACS column is then removed from the separator and placed on a suitable collection tube. 6 mL of buffer is loaded onto the MACS column and immediately thereafter, the bound Fc-LTM cells are flushed out through applying the column plunger. Low affinity or non-functional binding Fc-LTM variant cells are not retained in this manner.

This positive selection then recovers only those Fc-LTM variant cells with functional affinity to C1q/FcgRIIIa. This MACS enrichment step will eliminate the need of the FACS to process and sort unwanted cells. After elution, the enriched NS0 cells are then incubated for further culture (FIG. 4B).

### Example 7

### FACS Sorting of Fc-LTM Variant Library Cells

The following methodology involves FACS screening LTM Fc libraries for enrichment and isolation of FcR binding affinity variants. After growth culture, the above NS0 cells are incubated with biotinylated C1q at saturating concentrations (400 nM) for 3 hours at 37C under gentle rotation. (As before, biotinylated FcγRIIIa can be substituted for those appropriate experiments.) The NS0 cells are then twice washed with RPMI growth medium to remove unbound biotinylated C1q/FcγRIIIa. The cells are then sorted on FACS-Vantage (Becton Dickinson) using CellQuest software as per manufacturer's directions.

Depending on the binding characteristics desired, the sort gate and be adjusted to collect that fraction of the Fc-LTM population. For example, if enhanced affinity for FcγRIIIa is desired, the gate will be set for higher florescence signals. We have shown that FACS gating is able to enrich, by more than 80%, for a higher affinity sub-population in test system with other cell lines and associated binding proteins (FIG. 19).

### Example 8

### A. Fc Effector Functional Assays on Fc-LTM Cell Library

The following studies are performed to demonstrate that surface expression of Fc-LTM by NS0 cells that can lead to the engagement of FcγR on effector cells, such as monocytes and activated granulocytes, thereby initiating FcγR-dependent effector functions (FIG. 7: CDC, ADCC).

The FACS pre-sorted library is diluted into 96 well plates. Alternatively, after pLXSN/Fc transduction of NS0 cells, if only a small library is made (10⁶), these cells could also be directly plated at dilution of a single clone/well. These single clone wells can be then grown and expanded into daughter plates. One of these daughter plates can later serve as an Fc-effector assay plate. Thus, in some cases a small Fc-LTM library will not need the above MACS and/or FACS pre-sort.

It should be noted that in the following selection assays for higher affinity to Fc receptors C1q/FcγRIIIa and associated enhanced Fc effector C1q/FcγRIIIa functions, the additional step of screening for lower affinity to other Fc receptors such as FcγRIIb and diminished Fc effector functions can be performed in parallel (FIG. 5).

### B. Cell Dependent Cytotoxicity (CDC) Assay

Normal human mononuclear cells were prepared from heparinized bone marrow samples by centrifugation across a Ficoll-Hypaque density separation gradient. Human AB serum (Gemini Bioproducts, Woodland, Calif.) was used as the source of human complement., The ability of the NS0 library cells to promote complement mediated cytotoxicity was measured in an analogous manner. Briefly, the NS0 cells were cultured as above and plated (5×10⁴) were placed in 96-well flat-bottom microtiter wells. Human serum complement (Quidel, San Diego, Calif.) was serially diluted to first gauge a working range of lysis. The mixture of diluted complement and NS0 cell suspensions is then incubated for 2 h at 37° C. in a 5% CO₂ incubator to facilitate CDC. Afterwards, 50 μl of Alamar Blue (Accumed International, Westlake, Ohio) is added to each well and further incubated overnight at 37° C. Using a 96-well fluorometer, the fluorescence reading with excitation at 530 nm and emission at 590 nm is measured. Typically, the results are expressed in relative fluoresence units (RFU) in proportion to the number of viable cells. The activity of the various mutants is then examined by plotting the percent CDC activity against the log of Ab concentration (final concentration before the addition of Alamar Blue). The percent CDC activity was calculated as follows: % CDC activity=(RFU test−RFU background)×100 (RFU at total cell lysis —RFU background).

### C. Preparation of PBMC Effector Cells for ADCC

Effector PBMCs are prepared from heparinized whole venous blood from normal human volunteers. The whole blood is diluted with RPMI (Life Technologies, Inc.) containing 5% dextran at a ratio of 2.5:1 (v/v). The erythrocytes are then allowed to sediment for 45 minutes on ice, after which the cells in the supernatant are transferred to a new tube and pelleted by centrifugation. Residual erythrocytes are then removed by hypotonic lysis. The remaining lymphocytes, monocytes and neutrophils can be kept on ice until use in binding assays. Alternatively, effector cells can be purified from donors using Lymphocyte Separation Medium (LSM, Organon Technika, Durham, N.C.).

Target NS0 library cells expressing Fc variants are washed three times with RPMI 1640 medium and incubated with purified FcR (all types) at 1 mg/ml (concentration to be determined for maximum ADCC) for 30 min at 25° C. The above purified PBMC effector cells are washed three times with medium and placed in 96-well U-bottom Falcon plates (Becton Dickinson). To first gauge the working range of ADCC for these experiments, three-fold serial dilutions from 3×10⁵ cells/well (100:1 effector/target ratio) to 600 cells/well (0.2:1) are plated. Typically, ADCC is assayed in the presence of 50 fold excess of harvested PMBC.

Target NS0 cells are then added to each well at 3×103 cells/well. Spontaneous release (SR, negative control) is measured by NS0 target wells without added effector cells; conversely, maximum release (MR, positive control) is measured by adding 2% Triton X-100 to NS0 target cell wells. After 4 h of incubation at 37° C. in 5% CO2, ADCC assay plates are centrifuged. The supernatant are then transferred to 96-well flat-bottom Falcon plates and incubated with LDH reaction mixture (LDH Detection Kit, Roche Molecular Biochemicals) for 30 min at 25° C. The reactions are then stopped by adding 50 ml of 1 N HCl. After which, the samples are measured at 490 nm with reference wavelength of 650 nm. The percent cytotoxicity was calculated as [(LDH releaseₛₐₘₚₗₑ−SR_{effector}−SR_{target})/(MR_{target}−SR_{target})]×100. For each assay, the percent cytotoxicity versus log(effector/target ratio) is plotted and the area under the curve (AUC) calculated. The assays are performed in triplicate.

### Example 9

### Genotyping of PMBC Donors

### Screening of Fc□RIIIaF158/V158 Polymorphisms and FcγRIIa H131/R131 Polymorphism

For some experiments, as explained in the detailed description, we require monitoring the quantitative ADCC effector differences in between individuals with either FcγRIIIa F158/V158 and/or FcγRIIa H131/R131 polymorphisms. There are several ways to genotype the polymorphisms including; PCR followed by, direct sequencing, PCR using allele specific primers, or PCR followed by allele-specific restriction enzyme digestion. For our purposes, the latter allele-specific restriction enzyme digestion procedure for FcγRIIIa F158/V158 is described and the methodology is similar for FcγRIIa H131/R131 polymorphism (albeit using different PCR amplification primers).

Genotyping of the FcγRIIIA-158VWF polymorphism is performed by means of PCR-based allele-specific restriction analysis assay. Two FcγRIIIa gene-specific primers: 5′-ATA TTT ACA GAA TGG CAC AGG-3′; antisense SEQ ID: 5′-GAC TTG GTA CCC AGG TTG AA-3′; are used to amplify a 1.2-kb fragment containing the polymorphic site. This PCR assay was performed in buffer with 5 ng of genomic DNA, 150 ng of each primer, 200 μmol/L of each dNTP, and 2 U of Taq DNA polymerase (Promega, Madison, Wis.) as recommended by the manufacturer. The first PCR cycle consisted of 10 minutes denaturation at 95° C., 1½ minute primer annealing at 56° C., and 1½ minute extension at 72° C. This was followed by 35 cycles in which the denaturing time was decreased to 1 minute. The last cycle is followed by 8 minutes at 72° C. to complete extension. The sense primer in the second PCR reaction contains a mismatch that created an NIaIII restriction site only in FcγRIIIA-158V-encoding DNA: 5′-atc aga ttc gAT CCT ACT TCT GCA GGG GGC AT-3′; uppercase characters denote annealing nucleotides, lowercase characters denote nonannealing nucleotides), the antisense primer was chosen just 5′ of the fourth intron: 5′-acg tgc tga gCT TGA GTG ATG GTG ATG TTC AC-3′). This second PCR reaction is performed with 1 μL of the first amplified fragment, 150 ng of each primer, 200 μmol/L of each dNTP, and 2 U of Taq DNA polymerase, diluted in the recommended buffer. The first cycle consisted of 5 minutes' denaturing at 95° C., 1 minute primer annealing at 64° C., and 1 minute extension at 72° C. This was followed by 35 cycles in which the denaturing time was 1 minute. The last cycle was followed by 9½ minutes at 72° C. to complete extension. The 94-bp fragment was digested with NlaIII, and digested fragments were electrophoresed in 10% polyacrylamide gels, stained with ethidium bromide, and visualized with UV light.

FcγRIIa genotyping was determined using gene-specific sense: 5′-GGA AAA TCC CAG AAA TTC TCG C-3′; antisense SEQ ID: 5′-CAA CAG CCT GAC TAC CTA TTA CGCG GG-3′ primers. The sense primer is from the exon encoding the second extracellular domain upstream of codon 131 and ends immediately 5′ to the polymorphic site. It contains a one nucleotide substitution which introduces a Bst UI site (5′˜CGCG-3′) into the PCR product when the next nucleotide is G, but not when the next nucleotide is A. The antisense primer is located in the downstream intron and contains a two nucleotide substitution which introduces an obligate Bst Ul site into all PCR products which use this primer. The PCR conditions were as follows: one cycle at 96° C. for five minutes, 35 cycles at 92° C. for 40 seconds and 55° C. for 30 seconds, and one cycle at 72° C. for 10 minutes. Products were digested using Bst UI, which cuts once in the presence of the R131 allele and twice in the presence of the H131 allele. Fragments were resolved by electrophoresis on a 3% agarose gel.

### Example 10

### Construction of Full length Rituxin-Fc LTM Variant for Comparative ADCC and CDC Analysis

CBM-Fc or L™-Fc variants that exhibit the desired in vitro Fc receptor binding properties will then be tested for correlative Fc effector functions. For these assays we will compare the CBM-Fc or L™-Fc variant with the Rituxin Fc to determine if there are differences in ADCC and CDC activity. Developed for the treatment of non-Hodgkin's lymphoma, Rituxin is a chimeric monoclonal IgG, antibody specific for the B-cell marker CD20. For our purposes, we will compare wild type Rituxin (having the wild type IgG₁ Fc region) with chimeric Rituxin (CH1: V_{H} and V_{L}) and CBM-Fc or L™-Fc variant (hinge, C_{H}2 and C_{H}3) replacement.

By PCR with appropriate primers, the hinge, C_{H}2 and C_{H}3 will be amplified from CBM-Fc or L™-Fc variant. The primers will also introduce restriction sites into heavy-chain hinge and C_{H}3C-terminus for subsequent restriction digest and cloning. The Rituxin vector has been modified with similar restriction sites at the heavy-chain hinge region and C_{H}3C-terminus without changing to the amino-acid sequence. The modified Rituxin vector then allows simple replacement of the Fc domain while retaining its' V_{H} and V_{L} specificity for CD20.

After sequence verification, the Rituxin-Fc-LTM construct is re-cloned into PcDNA3 vector (Invitrogen) for expression as a soluble IgG₁. Briefly, the PcDNA3-Rituxin-Fc-LTM is transfected into CHO-K1 cells using lipofectamine (Invitrogen) and cultured in Dulbecco's modified Eagle's medium with 5% heat-inactivated fetal calf serum. If stable transfected clones are desired, they can then be selected with in the DMEM growth media with supplemented G418 (400 ug/ml). The supernatants from the above transfection are then collected, clarified by centrifugation to pellet all detached cells and debris. The secreted full length Rituxin-Fc-LTM IgG, can be purified by passing the culture supernatant over a Protein A Sepharose 4B affinity column. After washing with two to three column volumes of PBS, bound Rituxin-Fc-LTM IgG₁ protein is eluted with KSCN (3 M) in phosphate-buffered saline (10 mM sodium phosphate, 0.154 M NaCl, pH 7.3). Protein concentrations are estimated using absorbance at 280 nm and can be stored long term in phosphate-buffered saline (pH 7.3), containing sodium azide (0.8 mM) at −20° C.

The purified antibody is then added to WILS-2 target cells for ADCC, CDC or apoptosis assays. Apoptosis of WIL2-S cells can be analyzed by flow cytometric analysis using propidium iodide (PI; Molecular Probes, Eugene, Oreg.) and annexin V-FITC (Caltag, Burlingame, Calif.). Briefly, 5×105 WIL2-S cells are incubated with the specified concentrations of Rituxin wild type or Rituxin grafted Fc-LTM for 24 h at 37° C. and 5% CO₂. The target WIL2-S cells are then washed in PBS and resuspended in 400 ml of ice-cold annexin binding buffer (BD PharMingen, San Diego, Calif.) to which 10 ml of annexin V-FITC and 0.1 mg PI are added. Cells are then analyzed on a flow cytometer (Beckman-Coulter, Miami, Fla.): for excitation at 488 nm and measured emission at 525 nm (FITC) and 675 nm (PI) after compensation for overlapping emission spectra.

Although the invention has been described with respect to particular embodiments and applications, it will be appreciated that various modification and changes may be made without departing from the invention.

It is claimed:

1. A method of generating human Igd antibodies with enhanced effector function, comprising
(a) constructing an IgGi Fc look-through mutagenesis (LTM) coding library selected from one of:
(i) a regional LTM library encoding, for at least one of the two Igd Fc regions identified by SEQ ID NOS: 1 and 2, representing the CH2 and CH3 regions of the antibody's Fc fragment, respectively, and for each of a plurality of amino acids, individual amino acid substitutions at multiple amino acid positions within said at least one of the two IgGi Fc regions, and
(ii) a sub-region LTM library encoding, for each of the four regions identified by SEQ ID NOS: 3-6 contained within the IgGi Fc CH2 region identified by SEQ ID NO:1, and for each of a plurality of selected amino acids, individual substitutions at multiple amino acid positions within each region, and
(b) expressing the IgGi Fc fragments encoded by the LTM library in a selectable expression system, and
(c) selecting those IgGi Fc fragments expressed in (b) that are characterized by an enhanced effector function related to at least one of:
(i) a shift in binding affinity constant (Ko), with respect to a selected IgGt Fc binding protein, relative to native IgGt Fc; and
(ii) a shift in the binding off-rate constant (Koff); with respect to a selected IgGi Fc binding protein, relative to native IgGT Fc.

2. The method of claim 1, wherein the expressed Fc fragments encoded by said library are expressed in a selectable expression system having particles selected from the group consisting of viral particles, prokaryotic cells, and eukaryotic cells, and the expressed Fc particles are attached to the surface of the expression-system particles and accessible thereon to binding by said Fc binding protein.

3. The method of claim 2, wherein said expression system includes a mammalian cell that is (i) capable of producing clinical-grade monoclonal antibodies, (ii) nonadherent in culture, and (iii) readily transduced with retrovirus.

4. The method of claim 3, wherein said expression system cells are selected from the group consisting of BaF3, FDCP1, CHO, and NSO cells.

5. The method of claim 2, wherein said expression system includes a mammalian cell that expresses said Fc fragments on its surface, and step (c) includes (i) adding expression cells corresponding to a single clonal variant of said LTM library to each of a plurality of assay wells, (ii) adding to each well, reagents that include an Fc binding protein and which are effective to interact with said surface-attached Fc fragment, and depending on the level of binding thereto, to lyse said cells, (iii.) assaying the contents of said wells for the presence of cell lysis products, and (iv) selecting those IgG-i Fc fragments which are expressed on cells showing the greatest level of cell lysis.

6. The method of claim 5, wherein the reagents added in step (cii) are peripheral blood mononuclear cells capable of lysing cells expressing the Fc fragment on their surface by antibody-dependent cellular cytotoxicity.

7. The method of claim 6, wherein step (c) further includes, prior to step (ci), enriching such cells for those expressing Fc fragments having an elevated binding affinity constant or reduced binding off-rate constant, with respect to Fc-binding proteins FcyRI or FcvRIIIa.

8. The method of claim 5, wherein the reagents added in step (cii) are human C1q complex and human serum, capable of lysing cells by complementmediated cell death.

9. The method of claim 8, wherein step (c) further includes, prior to step (ci), enriching such cells for those expressing Fc fragments having an elevated binding affinity constant or reduced binding off-rate constant, with respect to Fcbinding protein C1q.

10. The method of claim 5, which further includes, prior to step (ci), enriching such cells for those expressing Fc fragments having one of:
(i) an elevated binding affinity constant or reduced binding off-rate constant, with respect to Fc-binding protein C1q, FcyRI, FcyRUa, and FcvRIIIa,
(ii) a reduced binding affinity constant or elevated binding off-rate constant with respect to Fc-binding proteins FcyRIIb, FcyRNIb; and an elevated or reduced binding affinity constant or a reduced or elevated binding off-rate constant, respectively, with respect to Fc-binding protein FcRN and protein A.

11. The method of claim 2, wherein said Fc fragments are selected for those having an elevated binding affinity constant, with respect to Fc-binding protein selected from the group consisting of C1q, FcvRI, FcyRIIa, FcyRIIIa, FcRN and protein, relative to the binding affinity constant for native IgGi Fc fragment, and step (c) includes {ci) forming a mixture of expression particles with displayed Fc fragments and an Fc binding protein, (cii) allowing the Fc receptor to bind with the displayed Fc fragments in the mixture, to form an Fc-binding complex, and (ciii) isolating said Fc-binding complexes from the mixture, wherein particles expressing Fc fragments having the highest binding affinity constants for said binding protein are isolated.

12. The method of claim 2, for selecting Fc fragments having an elevated equilibrium binding affinity constant, with respect to Fc-binding protein selected from the group consisting of C1q, FcyRI, FcyRIIa, FcyRNIa, FcRN and protein A, relative to the binding affinity constant for native IgGi Fc fragment, wherein step (c) includes (di) forming a mixture of expression particles with displayed Fc fragments and a limiting amount of fluorescent-labeled Fc binding protein in soluble form, such that those particles expressing Fc fragments with a higher binding affinity constant will be more strongly labeled, (cii) after the binding in the mixtures reaches equilibrium, sorting said particles on the basis of amount of bound fluorescent label, and (ciii), selecting those particles having the highest levels of bound fluorescence.

13. The method of claim 2, for selecting Fc fragments having a reduced binding off-rate constant, with respect to Fc-binding protein selected from the group consisting of FcyRIIb, FcvRMIb, FcRN and protein A, relative to the binding affinity constant for native IgG-i FC fragment, wherein step (c) includes—(ci) forming a mixture of expression particles with displayed Fc fragments and a limiting amount of fluorescent-labeled Fc binding protein in soluble form, such that those particles expressing Fc fragments with a lower binding affinity constant will be less strongly labeled, (cii) after the binding in the mixtures reaches equilibrium, sort said particles on the basis of amount of bound fluorescent label, and (ciii), selecting those particles having the lowest levels of bound fluorescence.

14. The method of claim 2, for selecting Fc fragments having an reduced binding off-rate affinity constant, with respect to Fc-binding protein selected from the group consisting of C1q, FcyRI, FcyRNa, FcyRIIIa, FcRN and protein A, relative to the binding affinity constant for native IgGj Fc fragment, wherein step (c) includes (ci) forming a mixture of expression particles with displayed Fc fragments and a saturating amount of fluorescent-labeled Fc binding protein in soluble form, (ii) at a selected time after step (ci), adding a saturating amount of an unlabeled Fc binding protein, (ciii) at a selected time after step (cii) and prior to binding equilibrium, sort said particles on the basis of amount of bound fluorescent label, and (civ), selecting those particles having the highest levels of bound fluorescence.

15. The method of claim 2, for selecting Fc fragments having an so increased binding off-rate affinity constant, with respect to Fc-binding protein selected from the group consisting of FeyRIib, FcyRIIIb, FcRN and protein A, relative to the binding affinity constant for native IgGi Fc fragment, wherein step (c) includes (ci) forming a mixture of expression particles with displayed Fc fragments and a saturating amount of fluorescent-labeled Fc binding protein in soluble form, (ii) at a selected time after step (ci), adding a saturating amount of an unlabeled Fc binding protein, (ciii) at a selected time after step (cii) and prior to binding equilibrium, sort said particles on the basis of amount of bound fluorescent label, and (civ), selecting those particles having the lowest levels of bound fluorescence.

16. The method of claim 1, for use in selecting Fc fragments having the ability, when incorporated into an IgGi antibody, to enhance antibody-dependent cellular-toxicity, which further includes, after identifying IgGi Fc fragments characterized by an elevated binding affinity constant or reduced binding off-rate, constant for FcyRIIIA, further selecting said identified fragments for binding affinity for the FcyRIIB receptor that exhibits reduced binding affi−nity constant or elevated binding off-rate constant for the FcyRIIB receptor.

17. The method of claim 1, for use in selecting Fc fragments having the ability, when incorporated into an IgGi antibody, to enhance complementdependent cytotoxicity (CDC), wherein step (c) further includes, after identifying IgGi Fc fragments characterized by an elevated binding affinity constant or reduced binding off-rate constant for C1 q complex, further selecting said identified fragments for binding affinity for the FcyRUB receptor that exhibits reduced binding affinity constant or elevated binding off-rate constant for the FcyRIIB receptor.

18. The method of claim 1, for use in selecting Fc fragments having the ability, when incorporated into an exogenous therapeutic IgGi antibody, to enhance the therapeutic response to the antibody in human patients having a position position-158 receptor polymorphism in the FcyRNIA receptor wherein so step (c) includes selecting those IgGi Fc fragments expressed in (b) that are characterized by a binding affinity for the FcyRIIIA F158 receptor polymorphism that is at least as great as that for a FcyRHIA V158 receptor polymorphism.

19. The method of claim 8, for use in selecting Fe fragments having the ability, when incorporated into an exogenous therapeutic Igd antibody, to enhance the therapeutic response to the antibody in human patients having a position-34 receptor polymorphism in the FcyRIIA receptor, wherein step (c) includes selecting those IgGi Fc fragments expressed in (b) that are characterized by a binding affinity for the FcvRIIA R131 receptor polymorphism that is at least as great as that for a FcyRIIA H131 receptor polymorphism.

20. The method of claim 1, which further includes
(d) constructing a walk-through mutagenesis (WTM) library encoding, for at least one of the Fc coding regions at which amino acid substitutions are made in the LTM library, the same amino acid substitution at multiple amino acid positions within that region, where the substituted amino acid corresponds to an amino acid variation found in at least one amino acid position of an Fc fragment selected in step (c);
(e) expressing the IgGi Fc fragments encoded by the WTM library in a selectable expression system, and
(f) selecting those IgGi Fc fragments expressed in (e) that are characterized by a desired shift in binding affinity constant or binding off-rate constant with respect to a selected IgGi Fc binding protein, compared with the same constant measured for a native Fc fragment.

21. The method of claim 1, wherein those IgGi Fc fragments expressed in claim 1 (b) and selected in step (c) are characterized by an increased binding affinity constant or reduced binding off-rate constant for a human IgGi Fc-binding protein, and where the shift in constant relative to the same constant measured for a native Fc fragment is greater than a factor of 1.5.

22. The method of claim 1, wherein those IgGi Fc fragments expressed in claim 1 (b) and selected in step (c) are characterized by an decreased binding affinity constant or increased binding off-rate constant for a human IgG-i Fc-binding protein, and where the shift in constant relative to the same constant measured for a native Fc fragment is greater than a factor of 1.5.

23. A method of performing multiple site-directed Kunkel mutagenesis on a single-stranded DMA, comprising
(a) hybridizing a plurality of mutagenic oligonucleotide(s) to a singlestranded linear DNA template having discreet nucleotide sequence regions complementary to discreet regions of said DMA template, thus to form a partial heteroduplex composed of the DNA template and a plurality of oligonucleotides to hybridized thereto,
(b) converting the partial heteroduplex to a full-length heteroduplex in which the plurality of hybridized oligonucleotides form a single strand complementary to the DNA template except at the regions where the oligonucleotides have introduced mutations into the template sequence, and
(c) removing the DNA template.
